(19) United States
(12) Patent Application Publication
(10) Pub. No.: US 2019/0154696 A1
(43) Pub. Date: May 23, 2019

(54) Colorimetric Labeling and Detection Methods and Compositions

(71) Applicant: LIFE TECHNOLOGIES CORPORATION, Carlsbad, CA (US)

(72) Inventors: Scott CLARKE, Eugene, OR (US); Kyle GEE, Springfield, OR (US); Carmen FINNESSY, Brevard, NC (US)

(21) Appl. No.: 15/753,454

(22) PCT Filed: Aug. 30, 2016
(86) PCT No.: PCT/US2016/049467

§ 371 Date: Feb. 19, 2018

Related U.S. Application Data
(60) Provisional application No. 62/212,376, filed on Aug. 31, 2015.

(51) Int. Cl. G01N 33/58; C12Q 1/6813; G01N 33/52

(52) U.S. Cl. CPC G01N 33/581; G01N 33/52; C12Q 1/6813

(57) ABSTRACT

The present disclosure provides methods, compositions and kits for measuring cellular nascent nucleic acid synthesis by colorimetric labeling of nucleic acid. The nucleic acid synthesis can be measured as cell proliferation, DNA or RNA synthesis, gene expression or apoptosis. Additionally, these methods may be used to screen compounds for their effect on cellular proliferation by treating cells or an organism with the test compound simultaneous to or before treatment with a competitive nucleoside analog.

### CROSS-REFERENCE

This application is a 371 National Stage application from PCT/US2016/049467, filed Aug. 30, 2016, which claims the benefit of U.S. Provisional Patent Application No. 62/212,376, filed Aug. 31, 2015, which disclosure is herein incorporated by reference in its entirety.

### FIELD

The present disclosure relates to methods for labeling nucleic acids and their use.

### BACKGROUND

Cell division and cell death play central roles in the proper development of multi-cellular organisms and in the homeostatic maintenance of tissues. Loss or reduction of cell proliferative capability and dysregulation of cell death are among the most important phenomena that characterize the aging process. Disruption of normal control of cell proliferation and cell death also underlies many pathological conditions including cancer, infectious diseases, vascular disorders and neurodegenerative diseases.

The most characteristic biochemical feature of cell division is DNA synthesis, which occurs essentially only during the S phase of the cell cycle. Accordingly, the most commonly used methods for the study of cell cycle, DNA synthesis and cell proliferation rely on incorporation of labeled biosynthetic precursors into the newly synthesized DNA of proliferating cells. In these methods, labeled DNA precursors (e.g., [³H]-thymidine, 5-bromo-2′-deoxyuridine (BrdU) or ethynyl-2′-deoxyuridine (EdU)) are added to cells during replication, and their incorporation into genomic DNA is quantified following incubation and sample preparation. Incorporated [³H]-thymidine is generally detected by autoradiography. Detection of incorporated BrdU is performed immunologically after sample denaturation to allow access of monoclonal antibodies, and the resulting BrdU-labeled cells are then analyzed by flow cytometry or microscopy.

Methods for detecting BrdU-labeled DNA or radioactively-labeled DNA are well known in the art. For example, cells containing BrdU-labeled DNA may be treated with an anti-BrdU monoclonal antibody followed by a fluorescently-labeled secondary antibody. The fluorescent label may then be visualized and quantified by standard techniques, including plate assays, fluorescence microscopy, imaging, high content screening, or flow cytometry. To study cellular proliferation of specific tissues, animals are administered (e.g., injected) labeled DNA precursors, sacrificed and the tissues are removed and fixed for microscopic analysis.

Although [³H]-thymidine and BrdU incorporation labeling methods have proven valuable for studying cell cycle kinetics, DNA synthesis and sister chromatid exchange, as well as for assessing cell proliferation of normal or pathological cells or tissues under different conditions, these methods exhibit several limitations. The most notable disadvantage of [³H]-thymidine incorporation results from the complications and risks of using radioactivity. In addition, autoradiography is labor-intensive and time-consuming. Furthermore, because both methods are sample destructive, quantification can be performed at only one predetermined time point, and continuous monitoring of a single sample is not possible. Additionally, in contrast to [³H]-thymidine autoradiography, BrdU immunohistochemistry is not stoichiometric. Thus, the intensity or extent of labeling is highly dependent on the conditions used for detection and does not necessarily reflect the magnitude of DNA replication. For this reason, BrdU labeling as a measure of cell division is especially vulnerable to misinterpretation.

Another method uses a stable isotope-mass spectrometric technique and resolves some of the problems associated with the [³H]-thymidine and BrdU incorporation methods. In this technique, the deoxyribose moiety of nucleotides in replicating DNA is labeled endogenously through the de novo nucleotide synthesis pathway by using stable isotope ²H- or ¹³C-labeled glucose. The isotopic enrichment of the DNA is then detected and quantified by gas chromatographic/mass spectrometric (GC/MS) analysis after isolation, denaturation and hydrolysis of genomic DNA and TMS (trimethylsilyl) derivation of the resulting deoxyribonucleosides. Although this method has several advantages including being safe for use in humans, it has disadvantages including that it involves a lengthy and destructive processing of the sample prior to detection.

Copper-catalyzed azide-alkyne cycloaddition (CuAAC) or “click” labeling of the nucleoside analog EdU was first introduced in 2007 where it has become a standard assay for fluorescence-based detection of S-phase proliferation, replacing antibody-based BrdU with a simpler and more rapid protocol yielding a brighter signal (U.S. Patent Publication No. 2011/0118142 and U.S. Pat. No. 7,910,335). The click reaction provides superb reaction kinetics, high specificity and bioorthogonality, and recent improvements have made these assays GFP and R-PE compatible. However, there are several disadvantages to the fluorescence-based EdU analysis including 1) the need for specialized detection equipment, 2) fading of the signal over time, 3) incompatibility of fluorescence staining with pathology stains and 4) high autofluorescence of tissue samples.

Clearly, improved nucleic acid labeling techniques are still needed for the study of cell cycle kinetics, DNA synthesis and cellular proliferation in vitro and in vivo. In particular, the development of techniques that are simple, rapid and sensitive that do not require extensive sample preparation and/or do not result in sample destruction remain highly desirable.

### SUMMARY

The present disclosure provides colorimetric-based click assays for use with bright field microscopy thereby providing images that are compatible with hematoxylin & eosin (H&E) staining and standard tissue staining protocols.

Certain embodiments of the present disclosure provide an adapter linker having structural formula (I):

Az-L-TM (I)

wherein, Az is an azide moiety, L is a spacer, and TM is a tetrazine functional group. In certain embodiments, the spacer comprises 1 to 20 PEG groups. In certain embodiments, the spacer comprises 2 to 10 PEG groups. In certain embodiments, the spacer comprises 4 PEG groups. In certain embodiments, the spacer comprises 2 PEG groups.

Certain embodiments of the present disclosure provide methods of labeling a nucleic acid polymer, the methods comprising:
a) contacting a sample with an effective amount of an alkynyl-modified nucleoside analogue, thereby forming an alkynyl-modified nucleic acid polymer;
b) contacting the alkynyl-modified nucleic acid polymer with an adapter linker of structural formula (I) under conditions such that the alkynyl moiety of the alkynyl-modified nucleic acid polymer forms a covalent link with the azide moiety of the adapter linker, thereby forming an adapter intermediate; and
c) contacting the adapter intermediate with a detectable label comprising a cycloalkene group under conditions such that a covalent link forms between the tetrazine functional group of the adapter intermediate and the cycloalkene group of the detectable label, thereby forming a labeled nucleic acid polymer.

In certain embodiments, the alkynyl-modified nucleoside analogue is an EdU or an EdC. In certain embodiments, the cycloalkene group is a trans-cycloalkene or a cyclopropene. In certain embodiments, the detectable label is a colorimetric label. In certain embodiments, the detectable label is horseradish peroxidase, alkaline phosphatase, beta-galactosidase, glucose oxidase or beta-lactamase. In certain embodiments, the horseradish peroxidase is a trans-cyclooctene-horseradish peroxidase conjugate.

In certain embodiments, the step of contacting the alkynyl-modified nucleic acid polymer with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the alkynyl-modified nucleic acid polymer with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the alkynyl-modified nucleic acid polymer with the adapter linker is performed in the presence of a copper chelator.

According to certain embodiments, the present disclosure provides methods of measuring cellular proliferation, the methods comprising:
a) contacting a cell with an effective amount of an alkynyl-modified nucleoside analogue, such that the alkynyl-modified nucleoside analogue is incorporated into DNA of the cell;
b) contacting the cell with an adapter linker of structural formula (I) under conditions such that the alkynyl moiety of the alkynyl-modified nucleoside analogue forms a covalent link with the azide moiety of the adapter linker, thereby forming an adapter intermediate;
c) contacting the cell with a detectable label comprising a cycloalkene group under conditions such that a covalent link forms between the tetrazine functional group of the adapter intermediate and the cycloalkene group of the detectable label; and
d) measuring the amount of detectable label incorporated into the DNA, wherein the amount of label indicates the extent of cellular proliferation.

In certain embodiments of the present disclosure, methods of measuring cellular proliferation in an organism are provided, the methods comprising:
a) administering to an organism an effective amount of an alkynyl-modified nucleoside analogue, such that the alkynyl-modified nucleoside analogue is incorporated into DNA of cells of the organism;
b) contacting at least one cell of the organism with an adapter linker of structural formula (I) under conditions such that the alkynyl moiety of the alkynyl-modified nucleoside analogue forms a covalent link with the azide moiety of the adapter linker, thereby forming an adapter intermediate;
c) contacting the at least one cell of the organism with a detectable label comprising a cycloalkene group under conditions such that a covalent link forms between the tetrazine functional group of the adapter intermediate and the cycloalkene group of the detectable label; and
d) measuring the amount of detectable label incorporated into the DNA, wherein the amount of label indicates the extent of cellular proliferation.

In certain embodiments, the alkynyl-modified nucleoside analogue is an EdU or an EdC. In certain embodiments, the cycloalkene group is a trans-cycloalkene or a cyclopropene. In certain embodiments, the detectable label is a colorimetric label. In certain embodiments, the colorimetric label is selected from horseradish peroxidase, alkaline phosphatase, beta-galactosidase, glucose oxidase or beta-lactamase. In certain embodiments, the horseradish peroxidase is a trans-cyclooctene-horseradish peroxidase conjugate. In certain embodiments, the cell is in a multi-well plate.

In certain embodiments, the step of contacting the cell with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the adapter linker is performed in the presence of a copper chelator.

According to certain embodiments of the present disclosure, methods for identifying an agent that perturbs cellular proliferation are provided, the methods comprising:
a) contacting a cell with a test agent;
b) contacting the cell with an effective amount of an alkynyl-modified nucleoside analogue, such that the alkynyl-modified nucleoside analogue is incorporated into DNA of the cell;
c) contacting the cell with an adapter linker of structural formula (I) under conditions such that the alkynyl moiety of the alkynyl-modified nucleoside analogue forms a covalent link with the azide moiety of the adapter linker, thereby forming an adapter intermediate;
d) contacting the cell with a detectable label comprising a cycloalkene group under conditions such that a covalent link forms between the tetrazine functional group of the adapter intermediate and the cycloalkene group of the detectable label;
e) measuring the amount of detectable label incorporated into the DNA, wherein the amount of label indicates the extent of cellular proliferation; and
f) identifying the test agent as an agent that perturbs cellular proliferation if the amount of label measured in step (e) is less than or greater than the amount of label measured in a control application in which the cell is not contacted with the test agent.

In certain embodiments, the present disclosure provides for methods of identifying an agent that perturbs cellular proliferation in an organism, the methods comprising:
a) exposing an organism to a test agent;
b) administering to the organism an effective amount of an alkynyl-modified nucleoside analogue, such that the alkynyl-modified nucleoside analogue is incorporated into DNA of cells of the organism;
c) contacting at least one cell of the organism with an adapter linker of structural formula (I) under conditions such that the alkynyl moiety of the alkynyl-modified nucleoside analogue forms a covalent link with the azide moiety of the adapter linker, thereby forming an adapter intermediate;
d) contacting the at least one cell of the organism with a detectable label comprising a cycloalkene group under conditions such that a covalent link forms between the tetrazine functional group of the adapter intermediate and the cycloalkene group of the detectable label;
e) measuring the amount of detectable label incorporated into the DNA, wherein the amount of label indicates the extent of cellular proliferation; and
f) identifying the test agent as an agent that perturbs cellular proliferation in the organism if the amount of label measured in step (e) is less than or greater than the amount of label measured in a control application in which the organism is not exposed to the test agent.

In certain embodiments, the alkynyl-modified nucleoside analogue is an EdU or an EdC. In certain embodiments, the cycloalkene group is a trans-cyclooctene or a cyclopentene. In certain embodiments, the detectable label is a colorimetric label. In certain embodiments, the colorimetric label is selected from horseradish peroxidase, alkaline phosphatase, beta-galactosidase, glucose oxidase or beta-lactamase. In certain embodiments, the horseradish peroxidase is a trans-cyclooctene-horseradish peroxidase conjugate. In certain embodiments, the cell is in a multi-well plate.

In certain embodiments, the step of contacting the cell with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the adapter linker is performed in the presence of a copper chelator.

According to certain embodiments of the present disclosure, methods of measuring cellular DNA synthesis are provided, the methods comprising:
a) contacting a cell with an effective amount of an alkynyl-modified nucleoside analogue, such that the alkynyl-modified nucleoside analogue is incorporated into DNA of the cell;
b) contacting the cell with an adapter linker of structural formula (I) under conditions such that the alkynyl moiety of the alkynyl-modified nucleoside analogue forms a covalent link with the azide moiety of the adapter linker, thereby forming an adapter intermediate;
c) contacting the cell with a detectable label comprising a cycloalkene group under conditions such that a covalent link forms between the tetrazine functional group of the adapter intermediate and the cycloalkene group of the detectable label; and
d) measuring the amount of detectable label incorporated into the DNA, wherein the amount of label indicates the extent of cellular DNA synthesis.

In certain embodiments, the method measures a change in cellular DNA synthesis. In certain embodiments, the alkynyl-modified nucleoside analogue is an EdU or an EdC. In certain embodiments, the cycloalkene group is a trans-cyclooctene or a cyclopentene. In certain embodiments, the detectable label is a colorimetric label. In certain embodiments, the colorimetric label is selected from horseradish peroxidase, alkaline phosphatase, beta-galactosidase, glucose oxidase or beta-lactamase. In certain embodiments, the horseradish peroxidase is a trans-cyclooctene-horseradish peroxidase conjugate. In certain embodiments, the cell is in a multi-well plate.

In certain embodiments, the step of contacting the cell with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the adapter linker is performed in the presence of a copper chelator.

In certain embodiments, the present disclosure provides for methods of measuring cellular RNA synthesis, the method comprising:
a) contacting a cell with an effective amount of an alkynyl-modified nucleoside analogue, such that the alkynyl-modified nucleoside analogue is incorporated into RNA of the cell;
b) contacting the cell with an adapter linker of structural formula (I) under conditions such that the alkynyl moiety of the alkynyl-modified nucleoside analogue forms a covalent link with the azide moiety of the adapter linker, thereby forming an adapter intermediate;
c) contacting the cell with a detectable label comprising a cycloalkene group under conditions such that a covalent link forms between the tetrazine moiety of the adapter intermediate and the cycloalkene group of the detectable label; and
d) measuring the amount of detectable label incorporated into the RNA, wherein the amount of label indicates the extent of cellular RNA synthesis.

In certain embodiments, the methods measure a change in cellular RNA synthesis. In certain embodiments, the alkynyl-modified nucleoside analogue is an EU or an EC. In certain embodiments, the cycloalkene group is a trans-cycloalkene or a cyclopropene. In certain embodiments, the detectable label is a colorimetric label. In certain embodiments, the colorimetric label is selected from horseradish peroxidase, alkaline phosphatase, beta-galactosidase, glucose oxidase or beta-lactamase. In certain embodiments, the horseradish peroxidase is a trans-cyclooctene-horseradish peroxidase conjugate. In certain embodiments, the cell is in a multi-well plate.

In certain embodiments, the step of contacting the cell with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the adapter linker is performed in the presence of a copper chelator.

According to certain embodiments of the present disclosure, methods for detecting apoptosis are provided, the methods comprising:
a) contacting a cell with an effective amount of an alkynyl-modified nucleoside analogue and a terminal deoxynucleotidyl transferase (TdT), such that the alkynyl-modified nucleoside analogue is incorporated into DNA of the cell;
b) contacting the cell with an adapter linker of structural formula (I) under conditions such that the alkynyl moiety of the alkynyl-modified nucleoside analogue forms a covalent link with the azide moiety of the adapter linker, thereby forming an adapter intermediate;
c) contacting the cell with a detectable label comprising a cycloalkene group under conditions such that a covalent link forms between the tetrazine functional group of the adapter intermediate and the cycloalkene group of the detectable label; and
d) measuring the amount of detectable label incorporated into the DNA, wherein the amount of label indicates the presence of apoptosis.

In certain embodiments, the alkynyl-modified nucleoside analogue is an EdUTP or an EdCTP. In certain embodiments, the cycloalkene group is a trans-cycloalkene or a cyclopropene. In certain embodiments, the detectable label is a colorimetric label. In certain embodiments, the colorimetric label is selected from horseradish peroxidase, alkaline phosphatase, beta-galactosidase, glucose oxidase or beta-lactamase. In certain embodiments, the horseradish peroxidase is a trans-cyclooctene-horseradish peroxidase conjugate. In certain embodiments, the cell is in a multi-well plate.

In certain embodiments, the step of contacting the cell with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the adapter linker is performed in the presence of a copper chelator.

According to certain embodiments of the present disclosure, kits are provided wherein the kits comprise:
an alkynyl-modified nucleoside analogue;
an adapter linker of structural formula (I);
a detectable label comprising a cycloalkene group; and
instructions for use according to the methods disclosed herein.

In certain embodiments, the kits further comprise a terminal deoxynucleotidyl transferase (TdT). In certain embodiments, the detectable label is a colorimetric label. In certain embodiments, the kits further comprise copper in the Cu(I) reduction state. In certain embodiments, the Cu(I) is a cuprous salt. In certain embodiments, the cuprous salt is a cuprous halide. In certain embodiments, the kits further comprise copper in the Cu(II) reduction state and a reducing agent. In certain embodiment, the Cu(II) is Cu(NO₃)₂, Cu(OAc)₂ or CuSO₄. In certain embodiments, the kits further comprise a copper chelator.

In certain embodiments, the kits are for labeling nucleic acid polymers. In certain embodiments, the kits are for measuring cellular nucleic acid synthesis. In certain embodiments, the kits are for measuring cellular proliferation. In certain embodiments, the kits are for identifying a test agent that perturbs cellular proliferation. In certain embodiments, the kits are for detecting apoptosis.

In certain embodiments of the present disclosure, methods for labeling nucleic acid polymers are provided, the methods comprising:
a) contacting a nucleic acid polymer with an effective amount of an alkynyl-modified nucleoside analogue, thereby forming an alkynyl-modified nucleic acid polymer;
b) contacting the alkynyl-modified nucleic acid polymer with an azide-modified fluorescent dye under conditions such that the alkynyl moiety of the alkynyl-modified nucleoside analogue forms a covalent link with the azide moiety of the fluorescent dye, thereby forming a fluorescent intermediate;
c) contacting the fluorescent intermediate with an anti-fluorescent dye antibody that binds to the azide-modified fluorescent dye, thereby forming an antibody-bound intermediate; and
d) contacting the cell with a secondary antibody conjugated to a detectable label, wherein the secondary antibody binds to the anti-fluorescent dye antibody, thereby forming a labeled nucleic acid polymer.

In certain embodiments of the present disclosure, methods for labeling nucleic acid polymers are provided, the methods comprising:
a) contacting a nucleic acid polymer with an effective amount of an alkynyl-modified nucleoside analogue, thereby forming an alkynyl-modified nucleic acid polymer;
b) contacting the alkynyl-modified nucleic acid polymer with an azide-modified fluorescent dye under conditions such that the alkynyl moiety of the alkynyl-modified nucleoside analogue forms a covalent link with the azide moiety of the fluorescent dye, thereby forming a fluorescent intermediate; and
c) contacting the cell with an anti-fluorescent dye antibody conjugated to a detectable label, wherein the antibody binds to the azide-modified fluorescent dye, thereby forming a labeled nucleic acid polymer.

In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the alkynyl-modified nucleic acid polymer with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the alkynyl modified nucleic acid polymer with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the alkynyl-modified nucleic acid polymer with the azide-modified fluorescent dye is performed in the presence of a copper chelator.

According to certain embodiments of the present disclosure, methods for measuring cellular proliferation are provided, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody that binds to the azide-modified fluorescent dye;
d) contacting the cell with a secondary antibody conjugated to a detectable label, wherein the secondary antibody binds to the anti-fluorescent dye antibody; and
e) measuring the amount of detectable label, wherein the amount of label indicates the extent of cellular proliferation.

In certain embodiments, methods are provided for measuring cellular proliferation, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody conjugated to a detectable label, wherein the antibody binds to the azide-modified fluorescent dye; and
d) measuring the amount of detectable label, wherein the amount of label indicates the extent of cellular proliferation.

According to certain embodiments of the present disclosure, methods for measuring cellular proliferation in an organism are provided, the methods comprising:
a) administering to an organism an effective amount of an alkynyl-modified nucleoside analogue, such that the alkynyl-modified nucleoside analogue is incorporated into DNA of cells of the organism;
b) contacting at least one cell of the organism with an azide-modified fluorescent dye under conditions such that the alkynyl moiety of the alkynyl-modified nucleoside analogue forms a covalent link with the azide moiety of the fluorescent dye;
c) contacting the at least one cell of the organism with an anti-fluorescent dye antibody that binds to the azide-modified fluorescent dye;
d) contacting the at least one cell of the organism with a secondary antibody that is conjugated to a detectable label, wherein the secondary antibody binds to the anti-fluorescent dye antibody; and
e) measuring the amount of detectable label, wherein the amount of label indicates the extent of cellular proliferation.

In certain embodiments, methods are provided for measuring cellular proliferation in an organism, the methods comprising:
a) administering to an organism an effective amount of an alkynyl-modified nucleoside analogue, such that the alkynyl-modified nucleoside analogue is incorporated into DNA of cells of the organism;
b) contacting at least one cell of the organism with an azide-modified fluorescent dye under conditions such that the alkynyl moiety of the alkynyl-modified nucleoside analogue forms a covalent link with the azide moiety of the fluorescent dye;
c) contacting the at least one cell of the organism with an anti-fluorescent dye antibody conjugated to a detectable label, wherein the antibody binds to the azide-modified fluorescent dye; and
d) measuring the amount of detectable label, wherein the amount of label indicates the extent of cellular proliferation.

In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the cell with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified fluorescent dye is performed in the presence of a copper chelator.

According to certain embodiments of the present disclosure, methods for identifying an agent that perturbs cellular proliferation are provided, the methods comprising:
a) contacting a cell with a test agent;
b) contacting the cell with an effective amount of an alkynyl-modified nucleoside analogue, such that the alkynyl-modified nucleoside analogue is incorporated into DNA of the cell;
c) contacting the cell with an azide-modified fluorescent dye under conditions such that the alkynyl moiety of the alkynyl-modified nucleoside analogue forms a covalent link with the azide moiety of the fluorescent dye;
d) contacting the cell with an anti-fluorescent dye antibody that binds to the azide-modified fluorescent dye;
e) contacting the cell with a secondary antibody that is conjugated to a detectable label, wherein the secondary antibody binds to the anti-fluorescent dye;
f) measuring the amount of detectable label, wherein the amount of label indicates the extent of cellular proliferation; and
g) identifying the test agent as an agent that perturbs cellular proliferation if the amount of label measured in step (f) is less than or greater than the amount of label measured in a control application in which the cell is not contacted with the test agent.

According to certain embodiments of the present disclosure, methods for identifying an agent that perturbs cellular proliferation are provided, the methods comprising:
a) contacting a cell with a test agent;
b) contacting the cell with an effective amount of an alkynyl-modified nucleoside analogue, such that the alkynyl-modified nucleoside analogue is incorporated into DNA of the cell;
c) contacting the cell with an azide-modified fluorescent dye under conditions such that the alkynyl moiety of the alkynyl-modified nucleoside analogue forms a covalent link with the azide moiety of the fluorescent dye;
d) contacting the cell with an anti-fluorescent dye antibody conjugated to a detectable label, wherein the antibody binds to the azide-modified fluorescent dye;
e) measuring the amount of detectable label, wherein the amount of label indicates the extent of cellular proliferation; and
f) identifying the test agent as an agent that perturbs cellular proliferation if the amount of label measured in step (e) is less than or greater than the amount of label measured in a control application in which the cell is not contacted with the test agent.

In certain embodiments of the present disclosure, methods for identifying an agent that perturbs cellular proliferation in an organism are provided, the methods comprising:
a) exposing an organism to a test agent;
b) administering to the organism an effective amount of an alkynyl-modified nucleoside analogue, such that the alkynyl-modified nucleoside analogue is incorporated into DNA of cells of the organism;
c) contacting at least one cell of the organism with an azide-modified fluorescent dye under conditions such that the alkynyl moiety of the alkynyl-modified nucleoside analogue forms a covalent link with the azide moiety of the fluorescent dye;
d) contacting the at least one cell of the organism with an anti-fluorescent dye antibody that binds to the azide-modified fluorescent dye;
e) contacting the at least one cell of the organism with a secondary antibody that is conjugated to a detectable label, wherein the secondary antibody binds to the anti-fluorescent dye antibody;
f) measuring the amount of detectable label, wherein the amount of label indicates the extent of cellular proliferation; and
g) identifying the test agent as an agent that perturbs cellular proliferation if the amount of label measured in step (f) is less than or greater than the amount of label measured in a control application in which the organism is not exposed to the test agent.

According to certain embodiments of the present disclosure, methods for identifying an agent that perturbs cellular proliferation in an organism are provided, the methods comprising:
a) exposing an organism to a test agent;
b) administering to the organism an effective amount of an alkynyl-modified nucleoside analogue, such that the alkynyl-modified nucleoside analogue is incorporated into DNA of cells of the organism;
c) contacting at least one cell of the organism with an azide-modified fluorescent dye under conditions such that the alkynyl moiety of the alkynyl-modified nucleoside analogue forms a covalent link with the azide moiety of the fluorescent dye;
d) contacting the at least one cell of the organism with an anti-fluorescent dye antibody conjugated to a detectable label, wherein the antibody binds to the azide-modified fluorescent dye;
e) measuring the amount of detectable label, wherein the amount of label indicates the extent of cellular proliferation; and
f) identifying the test agent as an agent that perturbs cellular proliferation if the amount of label measured in step (e) is less than or greater than the amount of label measured in a control application in which the organism is not exposed to the test agent.

In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the cell with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified fluorescent dye is performed in the presence of a copper chelator.

According to certain embodiments of the present disclosure, methods for measuring cellular DNA synthesis are provided, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody that binds to the azide-modified fluorescent dye;
d) contacting the cell with a secondary antibody that is conjugated to a detectable label, wherein the secondary antibody binds to the anti-fluorescent dye; and
e) measuring the amount of detectable label, wherein the amount of label indicates the extent of cellular DNA synthesis.

In certain embodiments provided herein, methods for measuring cellular DNA synthesis are provided, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody conjugated to a detectable label, wherein the antibody binds to the azide-modified fluorescent dye; and
d) measuring the amount of detectable label, wherein the amount of label indicates the extent of cellular DNA synthesis.

In certain embodiments, the methods measure a change in cellular DNA synthesis. In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the cell with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified fluorescent dye is performed in the presence of a copper chelator.

In certain embodiments provided herein, methods of measuring cellular RNA synthesis are provided, the methods comprising:
a) contacting a cell with an effective amount of an alkynyl-modified nucleoside analogue, such that the alkynyl-modified nucleoside analogue is incorporated into RNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody that binds to the azide-modified fluorescent dye;
d) contacting the cell with a secondary antibody that is conjugated to a detectable label, wherein the secondary antibody binds to the anti-fluorescent dye antibody; and
e) measuring the amount of detectable label, wherein the amount of label indicates the extent of cellular RNA synthesis.

In certain embodiments provided herein, methods of measuring cellular RNA synthesis are provided, the methods comprising:
a) contacting a cell with an effective amount of an alkynyl-modified nucleoside analogue, such that the alkynyl-modified nucleoside analogue is incorporated into RNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody conjugated to a detectable label, wherein the antibody binds to the azide-modified fluorescent dye; and
d) measuring the amount of detectable label, wherein the amount of label indicates the extent of cellular RNA synthesis.

In certain embodiments, the methods measure a change in cellular RNA synthesis. In certain embodiments, the alkynyl-modified nucleoside analogue is an EU or an EC. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the cell with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified fluorescent dye is performed in the presence of a copper chelator.

According to certain embodiments of the present disclosure, methods for detecting apoptosis are provided, the methods comprising:
a) contacting a cell with an effective amount of an alkynyl-modified nucleoside analogue and a terminal deoxynucleotidyl transferase (TdT), such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody that binds to the azide-modified fluorescent dye;
d) contacting the cell with a secondary antibody that is conjugated to a detectable label, wherein the secondary antibody binds to the anti-fluorescent dye; and
e) measuring the amount of detectable label, wherein the amount of label indicates the presence of apoptosis.

In certain embodiments provided herein, methods for detecting apoptosis are provided, the methods comprising:
a) contacting a cell with an effective amount of an alkynyl-modified nucleoside analogue and a terminal deoxynucleotidyl transferase (TdT), such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody conjugated to a detectable label, wherein the antibody binds to the azide-modified fluorescent dye; and
d) measuring the amount of detectable label, wherein the amount of label indicates the presence of apoptosis.

In certain embodiments, the alkynyl-modified nucleoside analogue is an EdUTP or an EdCTP. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the cell with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified fluorescent dye is performed in the presence of a copper chelator.

According to certain embodiments of the present disclosure, kits are provided, wherein the kits comprise:
an alkynyl-modified nucleoside analogue;
an azide-modified fluorescent dye;
an anti-fluorescent dye antibody that binds to the azide-modified fluorescent dye;
a secondary antibody that is conjugated to a detectable label, wherein the secondary antibody binds to the anti-fluorescent dye antibody; and
instructions for use according to the methods provided herein.

In certain embodiments, the kits further comprise a terminal deoxynucleotidyl transferase (TdT). In certain embodiments, the detectable label is a colorimetric label. In certain embodiments, the kits further comprise copper in the Cu(I) reduction state. In certain embodiments, the Cu(I) is a cuprous salt. In certain embodiments, the cuprous salt is a cuprous halide. In certain embodiments, the kits further comprise copper in the Cu(II) reduction state and a reducing agent. In certain embodiment, the Cu(II) is Cu(NO₃)₂, Cu(OAc)₂ or CuSO₄. In certain embodiments, the kits further comprise a copper chelator.

In certain embodiments, the kits are for labeling nucleic acid polymers. In certain embodiments, the kits are for measuring cellular nucleic acid synthesis. In certain embodiments, the kits are for measuring cellular proliferation. In certain embodiments, the kits are for identifying a test agent that perturbs cellular proliferation. In certain embodiments, the kits are for detecting apoptosis.

According to certain embodiments of the present disclosure, kits are provided, wherein the kits comprise:
an alkynyl-modified nucleoside analogue;
an azide-modified fluorescent dye;
an anti-fluorescent dye antibody conjugated to a detectable label, wherein the antibody binds to the azide-modified fluorescent dye; and
instructions for use according to the methods provided herein.

In certain embodiments, the kits further comprise a terminal deoxynucleotidyl transferase (TdT). In certain embodiments, the detectable label is a colorimetric label. In certain embodiments, the kits further comprise copper in the Cu(I) reduction state. In certain embodiments, the Cu(I) is a cuprous salt. In certain embodiments, the cuprous salt is a cuprous halide. In certain embodiments, the kits further comprise copper in the Cu(II) reduction state and a reducing agent. In certain embodiment, the Cu(II) is Cu(NO₃)₂, Cu(OAc)₂ or CuSO₄. In certain embodiments, the kits further comprise a copper chelator.

In certain embodiments, the kits are for labeling nucleic acid polymers. In certain embodiments, the kits are for measuring cellular nucleic acid synthesis. In certain embodiments, the kits are for measuring cellular proliferation. In certain embodiments, the kits are for identifying a test agent that perturbs cellular proliferation. In certain embodiments, the kits are for detecting apoptosis.

In certain embodiments of the present disclosure, methods for labeling nucleic acid polymers are provided, the methods comprising:
a) contacting a nucleic acid polymer with an effective amount of an alkynyl-modified nucleoside analogue, thereby forming an alkyne-modified nucleic acid polymer;
b) contacting the alkyne-modified nucleic acid polymer with an azide-modified biotin under conditions such that the alkynyl moiety of the alkynyl-modified nucleoside analogue forms a covalent link with the azide moiety of the biotin, thereby forming a biotin-modified intermediate; and
c) contacting the biotin-modified intermediate with a (strept)avidin conjugated to a detectable label, thereby forming a labeled nucleic acid polymer.

In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase and beta-lactamase.

In certain embodiments, the step of contacting the alkyne-modified nucleic acid polymer with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the alkyne-modified nucleic acid polymer with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the alkyne-modified nucleic acid polymer with the azide-modified biotin is performed in the presence of a copper chelator.

According to certain embodiments of the present disclosure, methods of measuring cellular proliferation are provided, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified biotin under conditions such that the alkynyl moiety of the alkynyl-modified nucleoside analogue forms a covalent link with the azide moiety of the biotin;
c) contacting the cell with a (strept)avidin conjugated to a detectable label; and
d) measuring the amount of detectable label, wherein the amount of label indicates the extent of cellular proliferation.

According to certain embodiments of the present disclosure, methods of measuring cellular proliferation in an organism are provided, the methods comprising:
a) administering to an organism an effective amount of an alkynyl-modified nucleoside analogue, such that the alkynyl-modified nucleoside analogue is incorporated into DNA of cells of the organism;
b) contacting at least one cell of the organism with an azide-modified biotin under conditions such that the alkynyl moiety of the alkynyl-modified nucleoside analogue forms a covalent link with the azide moiety of the biotin;
c) contacting the at least one cell of the organism with a (strept)avidin conjugated to a detectable label; and
d) measuring the amount of detectable label, wherein the amount of label indicates the extent of cellular proliferation.

In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase and beta-lactamase.

In certain embodiments, the step of contacting the cell with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified biotin is performed in the presence of a copper chelator.

According to certain embodiments of the present disclosure, methods for identifying an agent that perturbs cellular proliferation are provided, the methods comprising:
a) contacting a cell with a test agent;
b) contacting the cell with an effective amount of an alkynyl-modified nucleoside analogue, such that the alkynyl-modified nucleoside analogue is incorporated into DNA of the cell;
c) contacting the cell with an azide-modified biotin under conditions such that the alkynyl moiety of the alkynyl-modified nucleoside analogue forms a covalent link with the azide moiety of the biotin;
d) contacting the cell with a (strept)avidin conjugated to a detectable label;
e) measuring the amount of detectable label, wherein the amount of label indicates the extent of cellular proliferation; and
f) identifying the test agent as an agent that perturbs cellular proliferation if the amount of label measured in step (e) is less than or greater than the amount of label measured in a control application in which the cell is not contacted with the test agent.

According to certain embodiments of the present disclosure, methods for identifying an agent that perturbs cellular proliferation in an organism are provided, the methods comprising:
a) exposing an organism to a test agent;
b) administering to the organism an effective amount of an alkynyl-modified nucleoside analogue, such that the alkynyl-modified nucleoside analogue is incorporated into DNA of cells of the organism;
c) contacting at least one cell of the organism with an azide-modified biotin under conditions such that the alkynyl moiety of the alkynyl-modified nucleoside analogue forms a covalent link with the azide moiety of the biotin;
d) contacting the at least one cell of the organism with a (strept)avidin conjugated to a detectable label;
e) measuring the amount of detectable label, wherein the amount of label indicates the extent of cellular proliferation; and
f) identifying the test agent as an agent that perturbs cellular proliferation if the amount of label measured in step (e) is less than or greater than the amount of label measured in a control application in which the organism is not exposed to the test agent.

In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase and beta-lactamase.

In certain embodiments, the step of contacting the cell with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified biotin is performed in the presence of a copper chelator.

According to certain embodiments of the present disclosure, methods of measuring cellular DNA synthesis are provided, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified biotin under conditions such that the alkynyl moiety of the alkynyl-modified nucleoside analogue forms a covalent link with the azide moiety of the biotin;
c) contacting the cell with a (strept)avidin conjugated to a detectable label; and
d) measuring the amount of detectable label, wherein the amount of label indicates the extent of cellular DNA synthesis.

In certain embodiments, the methods measure a change in cellular DNA synthesis. In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase and beta-lactamase.

In certain embodiments, the step of contacting the cell with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified biotin is performed in the presence of a copper chelator.

According to certain embodiments of the present disclosure, methods of measuring cellular RNA synthesis are provided, the methods comprising:
a) contacting a cell with an effective amount of an alkynyl-modified nucleoside analogue, such that the alkynyl-modified nucleoside analogue is incorporated into RNA of the cell;
b) contacting the cell with an azide-modified biotin under conditions such that the alkynyl moiety of the alkynyl-modified nucleoside analogue forms a covalent link with the azide moiety of the biotin;
c) contacting the cell with a (strept)avidin conjugated to a detectable label; and
d) measuring the amount of detectable label, wherein the amount of label indicates the extent of cellular RNA synthesis.

In certain embodiments, the methods measure a change in cellular RNA synthesis. In certain embodiments, the alkynyl-modified nucleoside analogue is an EU or an EC. In certain embodiments, the detectable label is a colorimetric label. In certain embodiments, the detectable label is selected from horseradish peroxidase, alkaline phosphatase, beta-galactosidase, glucose oxidase and beta-lactamase.

In certain embodiments, the step of contacting the cell with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified biotin is performed in the presence of a copper chelator.

According to certain embodiments of the present disclosure, methods of detecting apoptosis are provided, the methods comprising:
a) contacting a cell with an effective amount of an alkynyl-modified nucleoside analogue and a terminal deoxynucleotidyl transferase (TdT), such that the alkynyl-modified nucleoside analogue is incorporated into DNA of the cell;
b) contacting the cell with an azide-modified biotin under conditions such that the alkynyl moiety of the alkynyl-modified nucleoside analogue forms a covalent link with the azide moiety of the biotin;
c) contacting the cell with a (strept)avidin conjugated to a detectable label; and
d) measuring the amount of detectable label, wherein the amount of label indicates the presence of apoptosis.

In certain embodiments, the alkynyl-modified nucleoside analogue is an EdUTP or an EdCTP. In certain embodiments, the detectable label is a colorimetric label. In certain embodiments, the detectable label is selected from horseradish peroxidase, alkaline phosphatase, beta-galactosidase, glucose oxidase and beta-lactamase.

In certain embodiments, the step of contacting the cell with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified biotin is performed in the presence of a copper chelator.

According to certain embodiments of the present disclosure kits are provided, wherein the kits comprise:
an alkynyl-modified nucleoside analogue;
an azide-modified biotin;
a (strept)avidin conjugated to a detectable label; and
instructions for use according to the methods provided herein.

In certain embodiments, the kits further comprise a terminal deoxynucleotidyl transferase (TdT). In certain embodiments, the detectable label is a colorimetric label. In certain embodiments, the kits further comprise copper in the Cu(I) reduction state. In certain embodiments, the Cu(I) is a cuprous salt. In certain embodiments, the cuprous salt is a cuprous halide. In certain embodiments, the kits further comprise copper in the Cu(II) reduction state and a reducing agent. In certain embodiment, the Cu(II) is Cu(NO₃)₂, Cu(OAc)₂ or CuSO₄. In certain embodiments, the kits further comprise a copper chelator.

In certain embodiments, the kits are for labeling nucleic acid polymers. In certain embodiments, the kits are for measuring cellular nucleic acid synthesis. In certain embodiments, the kits are for measuring cellular proliferation. In certain embodiments, the kits are for identifying a test agent that perturbs cellular proliferation. In certain embodiments, the kits are for detecting apoptosis.

### BRIEF DESCRIPTION OF THE FIGURES

FIG. 1 shows a schematic of colorimetric click labeling of nucleic acids according to certain methods of the present disclosure.

FIG. 2A and FIG. 2B show an exemplary workflow for colorimetric click labeling of nucleic acids using EdU according to certain methods provided herein (FIG. 2A) compared to a workflow for BrdU labeling of nucleic acids (FIG. 2B). Both workflows are for formalin-fixed paraffin-embedded (FFPE) samples.

FIG. 3 is a micrograph of “double-click” detection of rat intestine pulsed with EdU and stained with hematoxylin (20× objective) according to certain embodiments of the methods provided herein.

FIG. 4A is a micrograph showing fluorescent intermediate of an OREGON GREEN™ (Thermo Fisher Scientific, Waltham, Mass.) click reaction according to certain embodiments of the methods provided herein.

FIG. 4B is a micrograph showing conversion of the OREGON GREEN™ signal to DAB using a goat-anti-rabbit-HRP conjugate, according to certain embodiments of the methods provided herein.

FIG. 5A is a micrograph showing biotin-azide/streptavidin-HRP/DAB detection of EdU (brown) in rat tissue also stained with hematoxylin and eosin (H&E) according to certain embodiments of the methods provided herein.

FIG. 5B is a micrograph of mouse cardiac tissue showing biotin-azide/streptavidin-HRP/DAB detection of EdU (brown) in rat tissue also stained with Russell-Movat pentachrome stain according to certain embodiments of the methods provided herein.

FIG. 5C is a micrograph of rat intestine showing conversion of the OREGON GREEN™ signal to DAB using a goat-anti-rabbit-HRP conjugate, according to certain embodiments of the methods provided herein.

FIGS. 6A, 6B and 6C are micrographs comparing BrdU labeling of nucleic acids in rat mammary tissue (FIG. 6A) with colorimetric Click EdU labeling of nucleic acids in rat mammary tissue (FIGS. 6B and 6C) according to certain embodiments of the methods provided herein.

FIGS. 7A, 7B, 7C and 7D are micrographs comparing BrdU labeling of nucleic acids in rat intestinal tissue (FIGS. 7A and 7B) with colorimetric click EdU labeling of nucleic acids in rat intestinal tissue (FIGS. 7C and 7D) according to certain embodiments of the methods provided herein.

FIGS. 8A, 8B, 8C, 8D and 8E are micrographs showing a TUNEL assay in mouse intestine according to certain embodiments of the methods provided herein. FIGS. 8A and 8B are micrographs showing biotin-azide streptavidin-HRP DAB detection of apoptotic cells using a TUNEL assay in mouse intestine treated with and without DNase (FIGS. 8A and 8B, respectively). FIG. 8C is a micrograph of mouse intestine stained with H&E only. FIGS. 8D and 8E are micrographs of different magnification showing biotin-azide streptavidin-HRB DAB detection of apoptotic cells (nuclei: brown, circled) in a sample of formalin-fixed paraffin embedded (FFPE) mouse intestines that were counter stained with methyl green (FIG. 8D is 20× magnification, FIG. 8E is 40× magnification).

FIG. 9 shows an exemplary workflow for colorimetric Click TUNEL labeling of apoptotic cells according to certain methods provided herein. The workflow is for FFPE samples.

### DETAILED DESCRIPTION

Introduction:

Herein we describe methods for measuring cellular nucleic acid synthesis with the incorporation of bio-orthogonal nucleoside or nucleotide analogs, including but not limited to azido-modified analogs, alkyne-modified analogs (such as EdU) or phosphine-modified analogs, such that the newly synthesized cellular nucleic acid can be labeled with colorimetrically detectable labels resulting in a significantly reduced workflow.

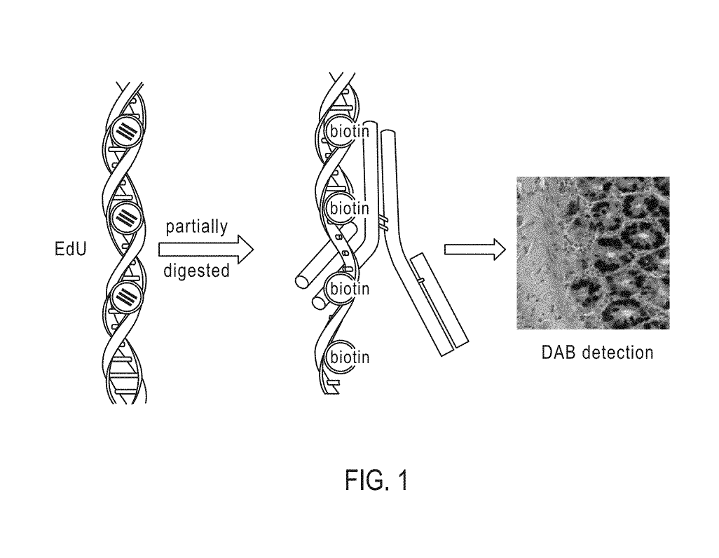

Detection of the thymidine analog EdU in tissue has been demonstrated using copper-catalyzed azide-alkyne cycloaddition (CuAAC) to covalently react azide-modified fluorescent dyes with DNA-incorporated EdU. Modification of the fluorescence labeling protocol can be used to sensitively detect EdU with chromophores visible by white light microscopy. Three approaches described herein were used to demonstrate the results of white light detection of EdU using modified click chemistry reagents. In certain exemplary embodiments, the colorimetrically detectable label comprises horseradish peroxidase (HRP) as the enzyme used for conversion of chromogenic substrates to insoluble products such as 3,3′-diaminobenzidine (DAB); however, the methods described herein are not limited to the use of HRP (see, FIG. 1). Other reporter enzymes can be used in the methods described herein, such as, but not limited to, alkaline phosphatase, beta-galactosidase, glucose oxidase or beta-lactamase.

1. “Double Click Reaction”:

In certain embodiments, the methods for labeling nucleic acids provided herein comprise two successive click reactions. The first reaction comprises a copper-catalyzed click reaction for the coupling of a heterobifunctional adapter linker of structural formula (I):

Az-L-TM (I)

wherein, Az is an azide moiety, L is a spacer, and TM is a tetrazine functional group, to an alkyne-modified nucleoside, such as EdU, that has been incorporated into the nucleic acid. The utility of this first step is twofold: 1) without wishing to be bound by theory, the side reaction of the copper-based click reaction results in improved access to tightly bound DNA through a Fenton-type scission reaction thereby improving accessibility to the incorporated modified nucleoside; 2) this copper-based click reaction attaches a tetrazine functional group to the alkyne-modified nucleoside by way of the heterobifunctional adapter linker of structural formula (I) consisting of an azide moiety at one end separated by a spacer to a tetrazine functional group at the other end. The second click reaction uses a copper-free Diels-Alder type of click reaction between the covalently linked tetrazine functional group linked to the alkyne-modified nucleoside and a colorimetrically detectable label comprising a cyclooctene, such as a trans-cyclooctene-HRP conjugate. The purpose of the second reaction is to attach the colorimetric detectable label, for example, the enzyme horseradish peroxidase (HRP), to the incorporated alkynyl-modified nucleoside, for example EdU. The use of a copper-free second reaction is important to maintain the catalytic activity of the detectable label, in this example HRP enzyme. The adapter linker of structural formula (I) serves to separate the alkynyl-modified nucleoside from the detectable label. It was unexpectedly found that HRP directly coupled to EdU has poor activity and is an inefficient reaction.

The advantages of the “Double Click Reaction” provided herein over currently available methods are 1) its simplicity and 2) eliminating the need for an antibody to attach the detectable label to the modified nucleoside. The workflow of the “Double Click Reaction” provided herein is simplified because the method has a single detection reaction. Furthermore, it allows for multiplexing with other labeling methods where multiple fluorescent antibodies can be used without the restriction of the antibody-host cross-reactivity to consider. For example, EdU can be detected with the double-click reaction and any antibody host species, such as mouse or rabbit, can be used to detect another epitope of interest.

2. Copper-Based Click Reaction of a Fluorescent Azide Intermediate (“Fluorescent Intermediate Method”):

In certain embodiments, the methods provided herein use a click reaction between an alkynyl-modified nucleoside and an azide-modified fluorescently labeled dye. The incorporated alkyne-modified nucleoside is coupled to the azide-modified fluorescent dye, such as an azide-modified OREGON GREEN™ dye. Subsequently, the fluorescently-labeled modified nucleic acid is incubated with an antibody that binds to the azide-modified fluorescent dye, for example, an anti-OREGON GREEN™ antibody, that is either 1) directly or indirectly conjugated to a colorimetrically detectable label, such as HRP, or 2) followed by an additional step of incubation with a secondary antibody conjugated to a colorimetric detectable label, such as an anti-fluorescent dye antibody-HRP conjugate. Use of a secondary antibody such as a goat-anti-rabbit HRP conjugate allows for additional signal amplification thereby improving sensitivity. This method is not restricted to the use of azide-modified fluorescent dyes, but can be extended to other azide-modified haptens such as digoxigenin or biotin which have good binding partners or antibodies against them.

An unexpected advantage of the “Fluorescent Intermediate Method” provided herein over currently available methods is that a fluorescent intermediate of the reaction can be viewed prior to coupling of the dye to the detectably labeled antibody conjugate. This provides a quick check of the reaction product while still continuing the development of color staining. Colorimetric staining is a standard for clinicians as it can be viewed in morphological context with stains such as hematoxylin and eosin (H&E). Another advantage of the method provided herein over currently available methods is that the colorimetric staining provides a permanent archival record.

3. Copper-Based Click Reaction of a Biotin Azide (“Biotin Intermediate Method”):

In certain embodiments, methods are provided that use two highly efficient reactions resulting in effective detection of alkyne-modified nucleosides, such as EdU. The first reaction comprises a copper-based click reaction using an azide-modified biotin that binds to an alkynyl-modified nucleoside. The second reaction comprises a conjugation reaction between the azide-modified biotin and a (strept)avidin bound to a colorimetrically detectable label, wherein a biotin-(strept)avidin complex is formed. Both of these reactions are efficient, rapid and essentially irreversible, resulting in a bright and sensitive signal in relatively few steps.

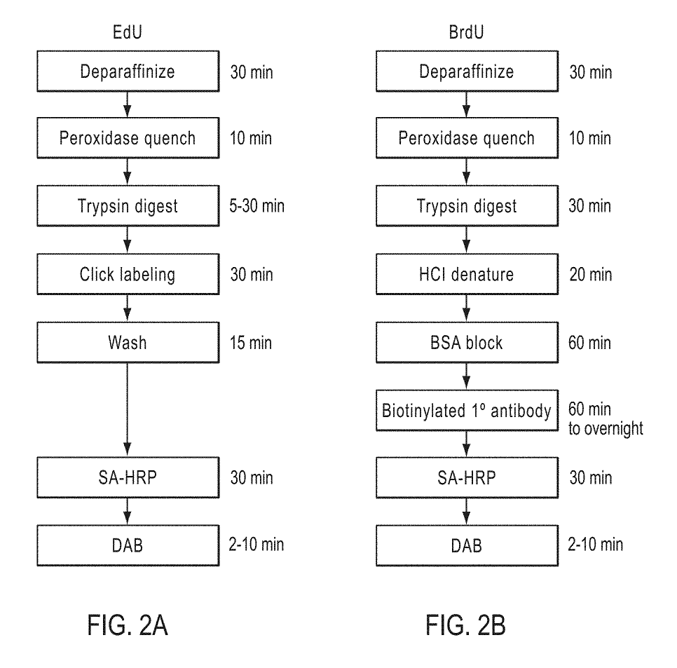

Advantages of the “Biotin Intermediate Method” provided herein over currently available methods include: 1) no antibody blocking steps are needed, 2) a rapid time to achieve results and 3) having two essentially irreversible steps (see, FIG. 2A). In addition, the small size of the azide-modified biotin allows for efficient detection of the incorporated alkyne-modified nucleoside using mild conditions and deparaffinization is sufficient for the azide-modified biotin to gain access to the nucleic acid polymer. However, trypsin digestion is required for the (strept)avidin to access the biotin-tagged EdU (see, FIG. 2A). This is in contrast to BrdU assays which require harsh methods such as HCl denaturation and/or heat inactivation epitope retrieval (HIER) (see, FIG. 2B).

Definitions:

Before describing the present teachings in detail, it is to be understood that this disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a ligand” includes a plurality of ligands and reference to “a nucleic acid” includes a plurality of nucleic acids and the like.

Unless defined otherwise, all technical and scientific terms used herein have the same meaning as commonly understood by one of ordinary skill in the art to which this disclosure is related. It is also understood that when describing chemical moieties or molecules that are attached to another compound, these moieties exist in a radical form for the purposes of conjugation. The following terms are defined for purposes of the disclosure as described herein.

As used herein, the term “alkyne-reactive” refers to a chemical moiety that selectively reacts with an alkyne-modified group on the nucleoside analog to form a covalent chemical link between the alkyne-modified group and the alkyne-reactive group. Examples of alkyne-reactive groups include azides. “Alkyne-reactive” can also refer to a molecule that contains a chemical moiety that selectively reacts with an alkyne group.

As used herein, the term “azide-reactive” refers to a chemical moiety that selectively reacts with an azido-modified group on another molecule to form a covalent chemical link between the azido-modified group and the azide-reactive group. Examples of azide-reactive groups include alkynes (e.g., terminal alkynes, activated alkynes, cycloalkynes) and phosphines (e.g. triaryl phosphine). “Azide-reactive” can also refer to a molecule that contains a chemical moiety that selectively reacts with an azido group.

As used herein the term “bioorthogonal chemical reporter” or “bioorthogonal labeling reagent” means a detectable label that comprises a chemical handle that will react selectively with the present nucleoside analog once incorporated into nucleic acid to form a covalent link.

As used herein, the term “cell” in the context of the applications of the present disclosure is meant to encompass eukaryotic and prokaryotic cells of any genus or species, with mammalian cells being of particular interest. “Cell” is also meant to encompass both normal cells and diseased cells, e.g., cancerous cells.

The terms “cell proliferation” and “cellular proliferation” are used herein interchangeably and refer to an expansion of a population of cells by the division of single cells into daughter cells, or to the division of a single cell to daughter cells.

The terms, “chemical handle” and “bioorthogonal moiety” as used herein refer to a specific functional group, such as an azide, alkyne, activated alkyne, phosphite, phosphine, and the like. The chemical handle is distinct from biological reactive groups, defined below, in that the chemical handles are moieties that are rarely found in naturally-occurring biomolecules and are chemically inert towards biomolecules (e.g., native cellular components), but when reacted with an azide- or alkyne-reactive group the reaction can take place efficiently under biologically relevant conditions (e.g., cell culture conditions, such as in the absence of excess heat or harsh reactants).

As used herein, the term “click chemistry” refers to the copper-catalyzed version of a [3+2] cycloaddition reaction between a first reactive unsaturated group on the incorporated nucleoside analog (or nucleotide analog) or labeling reagent and a second reactive unsaturated group present on the labeling regent or nucleoside analog (or nucleotide analog). This click chemistry reaction is described by Sharpless et al. (Sharpless et al., Angew Chem., Int. Ed. Engl., 41:1596-1599 (2002)).

As used herein, the term “copper (I) catalyst” or “Cu(I) catalyst” refers to a compound, molecule or reagent that catalyzes the [3+2] cycloaddition reaction between a first reactive unsaturated group on the incorporated nucleoside analog (nucleotide analog) or labeling reagent and a second reactive unsaturated group present on the labeling reagent or nucleoside analog (nucleotide analog). The term “copper (I) catalyst” includes exogenous copper (I) as well as copper chelating moieties. The term “copper chelating moieties” refers to any compound, molecule or reagent characterized by the presence of two or more polar groups that can participate in the formation of a complex with copper (I) ions.

As used herein, the term “copperless click chemistry” refers to a strain-promoted [3+2] cycloaddition reaction that can be carried out under physiological conditions, as described by Bertozzi et al. U.S. Patent Application Publication No. 2006/0110782; Baskin et al., Proc. Natl. Acad. Sci. USA, 104:16793-7 (2007); Agard et al., J. Am. Chem. Soc., 126:15046-7 (2004). The reaction is accomplished through use of a first molecule comprising a strained cycloalkyne moiety and a second molecule comprising an azide moiety. The azide moiety on the second molecule reacts, in the absence of a catalyst, with the strained cycloalkyne moiety on the first molecule, forming a final conjugate product comprising fused azide/cycloalkyne ring.

As used herein, the term “detectable response” refers to an occurrence of or a change in, a signal that is directly or indirectly detectable either by observation or by instrumentation. Typically, the detectable response is an optical response resulting in a change in the wavelength distribution patterns or intensity of absorbance or fluorescence or a change in light scatter, fluorescence lifetime, fluorescence polarization, or a combination of the above parameters.

As used herein, the term “dye” refers to a compound that emits light to produce an observable detectable signal.

As used herein, the terms “azide-modified dye” and “azide-modified biotin” refer to a dye or biotin with a reactive azide group, respectively.

As used herein, the term “cycloalkyne” refers to compounds or molecules which may be used in strained [3+2] cycloaddition reactions in order to label DNA. In this context, examples of cycloalkynes include, but are not limited to: cyclooctynes, difluorocyclooctynes, heterocycloalkynes, dichlorocyclooctynes, dibromocyclooctynes, or diiodocyclooctynes.

As used herein, the term “dual labeling” refers to a labeling process in which a nucleic acid polymer is labeled with two detectable agents that produce distinguishable signals. The nucleic acid polymer resulting from such a labeling process is said to be dually labeled.

As used herein, the term “differential labeling” refers to a labeling process in which two nucleic acid polymers are labeled with two detectable agents that produce distinguishable signals (i.e., a first nucleic acid polymer is labeled with a first detectable agent, a second nucleic acid polymer is labeled with a second detectable agent, and the first and second detectable agents produce distinguishable signals). The detectable agents may be of the same type or of different types.

As used herein, the term “effective amount” refers to the amount of a substance, compound, molecule, agent or composition that elicits the relevant response in a cell, a tissue, or an organism. For example, in the case of cells contacted with a nucleoside analog, an effective amount is an amount of nucleoside that is incorporated into the DNA of the cells.

As used herein, the term “fluorophore” or “fluorogenic” refers to a composition that demonstrates a change in fluorescence upon binding to a biological compound or analyte interest. Preferred fluorophores of the present disclosure include fluorescent dyes having a high quantum yield in aqueous media. Exemplary fluorophores include xanthene, indole, borapolyazaindacene, furan and benzofuran, cyanine among others. The fluorophores of the present disclosure may be substituted to alter the solubility, spectral properties or physical properties of the fluorophore.

As used herein, the terms “label” and “reporter molecule” refer to a chemical moiety or protein that retains its native properties (e.g. spectral properties, conformation and activity) when part of a labeling reagent of the present disclosure and used in the present methods. Illustrative labels or reporter molecules can be directly detectable (e.g., a fluorophore or chromogen) or indirectly detectable (e.g., a hapten or enzyme). Such labels include, but are not limited to, radio-labels that can be measured with radiation-counting devices; pigments, dyes or other chromogens that can be visually observed or measured with a spectrophotometer; spin labels that can be measured with a spin label analyzer; and fluorescent moieties, where the output signal is generated by the excitation of a suitable molecular adduct and that can be visualized by excitation with light that is absorbed by the dye or can be measured with standard fluorometers or imaging systems, for example. The label can be a luminescent substance such as a phosphor or fluorogen; a bioluminescent substance; a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The label may also take the form of a chemical or biochemical, or an inert particle, including but not limited to colloidal gold, microspheres, quantum dots, or inorganic crystals such as nanocrystals or phosphors (see, e.g., Beverloo, et al., Anal. Biochem. 203:326-34 (1992)). The terms “label” or “reporter molecule” can also refer to a “tag” or hapten that can bind selectively to a labeled molecule such that the labeled molecule, when added subsequently, is used to generate a detectable signal. For instance, one can use biotin, iminobiotin or desthiobiotin as a tag and then use an avidin or streptavidin conjugate of horseradish peroxidase (HRP) to bind to the tag, and then use a chromogenic substrate (e.g., tetramethylbenzidine) or a fluorogenic substrate such as AMPLEX™ Red or AMPLEX™ Gold (Thermo Fisher Scientific, Waltham, Mass.) to detect the presence of HRP. In a similar fashion, the tag can be a hapten or antigen (e.g., digoxigenin), and an enzymatically, fluorescently, or radioactively labeled antibody can be used to bind to the tag. Numerous labels are known by those of skill in the art and include, but are not limited to, particles, fluorescent dyes, haptens, enzymes and their chromogenic, fluorogenic, and chemiluminescent substrates, and other reporter molecules that are described in Molecular Probes Handbook of Fluorescent Probes and Research Chemicals by Richard P. Haugland, 10^{th} Ed., (2005), the contents of which are incorporated by reference, and in other published sources. As used herein a label or reporter molecule is not an amino acid.

The term “linker” or “L”, as used herein, refers to a single covalent bond or a series of stable covalent bonds incorporating 1-30 nonhydrogen atoms selected from the group consisting of C, N, O, S and P. In addition, the linker covalently attaches a carrier molecule or solid support to the present azido or activated alkyne modified nucleotides or nucleic acid polymers. Exemplary linking members include a moiety that includes —C(O)NH—, —C(O)O—, —NH—, —S—, —O—, and the like. A “cleavable linker” is a linker that has one or more cleavable groups that may be broken by the result of a reaction or condition. The term “cleavable group” refers to a moiety that allows for release of a portion, e.g., a reporter molecule, carrier molecule or solid support, of a conjugate from the remainder of the conjugate by cleaving a bond linking the released moiety to the remainder of the conjugate. Such cleavage is either chemical in nature or enzymatically mediated. Exemplary enzymatically cleavable groups include natural amino acids or peptide sequences that end with a natural amino acid. In addition to enzymatically cleavable groups, it is within the scope of the present disclosure to include one or more sites that are cleaved by the action of an agent other than an enzyme. Exemplary non-enzymatic cleavage agents include, but are not limited to, acids, bases, light (e.g., nitro benzyl derivatives, phenacyl groups, benzoin esters), and heat. Many cleavable groups are known in the art. See, for example, Jung et al., Biochem. Biophys. Acta, 761: 152-162 (1983); Joshi et al., J Biol. Chem., 265: 14518-14525 (1990); Zarling et al., J Immunol., 124: 913-920 (1980); Bouizar et al., Eur. J Biochem., 155: 141-147 (1986); Park et al., J Biol. Chem., 261: 205-210 (1986); Browning et al., J Immunol., 143: 1859-1867 (1989). Moreover a broad range of cleavable, bifunctional (both homo- and hetero-bifunctional) spacer arms are commercially available. An exemplary cleavable group, an ester, is cleavable group that may be cleaved by a reagent, e.g. sodium hydroxide, resulting in a carboxylate-containing fragment and a hydroxyl-containing product.

As used herein, the term “labeling reagent” refers to a reagent used to label and detect the incorporated nucleotide analog.

As used herein, the terms “nucleoside analog” and “nucleotide analog” are used interchangeably and refer to a molecule or compound that is structurally similar to a natural nucleoside or nucleotide that is incorporated into newly synthesized nucleic acid. In the case of nucleosides, once inside the cells, they are phosphorylated into nucleotides and then incorporated into nascent nucleic acid polymers. Nucleotides are difficult to get across the cell membrane due to their charges and are more labile than nucleosides, thus their use typically requires an additional step and reagents for transfection to transport the nucleotides across the lipid bilayer. The present nucleoside analogs are incorporated into nucleic acid polymers (e.g., DNA or RNA) in a similar manner as a natural nucleoside wherein the correct polymerase enzyme recognizes the analogs as natural nucleosides and there is no disruption in synthesis. These analogs comprise a number of different moieties which are ultimately used for detection, such as those that comprise a bioorthogonal moiety such as azido, alkyne or phosphine.

As used herein, the term “reactive group” refers to a group that is capable of reacting with another chemical group to form a covalent bond, i.e. is covalently reactive under suitable reaction conditions, and generally represents a point of attachment for another substance. As used herein, reactive groups refer to chemical moieties generally found in biological systems and react under normal biological conditions, and are herein distinguished from the chemical handle or bioorthogonal functional moiety, defined above, such as the azido and activated alkyne moieties of the present disclosure. As referred to herein the reactive group is a moiety, such as carboxylic acid or succinimidyl ester, that is capable of chemically reacting with a functional group on a different compound to form a covalent linkage. Reactive groups generally include nucleophiles, electrophiles and photoactivatable groups.

As used herein, the term “Staudinger ligation” refers to a chemical reaction developed by Saxon and Bertozzi (Science, 287:2007-2010 (2000)) that is a modification of the classical Staudinger reaction. The classical Staudinger reaction is a chemical reaction in which the combination of an azide with a phosphine or phosphite produces an aza-ylide intermediate, which upon hydrolysis yields a phosphine oxide and an amine. A Staudinger reaction is a mild method of reducing an azide to an amine; and triphenylphosphine is commonly used as the reducing agent. In a Staudinger ligation, an electrophilic trap (usually a methyl ester) is appropriately placed on a triarylphosphine aryl group (usually ortho to the phosphorus atom) and reacted with the azide, to yield an aza-ylide intermediate, which rearranges in aqueous media to produce a compound with amide group and a phosphine oxide function. The Staudinger ligation is so named because it ligates (attaches/covalently links) the two starting molecules together, whereas in the classical Staudinger reaction, the two products are not covalently linked after hydrolysis.

As used herein, the term “(strept)avidin” refers to both avidin and streptavidin.

As used herein, the terms “test agent”, “test compound” and “test treatment” refer to any substance, compound, molecule, agent, composition, or treatment, which is tested during the claimed methods for its effect on cellular proliferation or the cell cycle. The effect on cellular proliferation of these “test agents”, “test compounds” and “test treatments” is not limited by outcome, that is, they may increase, decrease or not affect cellular proliferation or the cell cycle.

Nucleoside and Nucleotide Analogs:

Both nucleoside and nucleotide analogs can be used in the present methods for measuring nascent nucleic acid synthesis. Nucleosides are typically used in experiments wherein the analogs are added to cell culture or administered to animals because the nucleoside analogs are easily taken up by live cells, wherein they are phosphorylated into a nucleotide and then incorporated into a growing nucleic acid polymer. In contrast nucleotides are labile and prone to enzyme cleavage, either before or after incorporation into cells, and are generally less stable than nucleosides. In addition, due to the additional charges from the phosphate groups, nucleotides are not easily transported into live cells and generally require a transfection step to get a sufficient concentration of nucleotides across the cellular membrane. This is not ideal for either in vivo or ex vivo/in vivo experiments where cell perturbation should be kept to a minimum to accurately interpret results. For these reasons, the following disclosure generally refers to nucleosides as the analog that is added to cells or animals, however this in no way is intended to be limiting, wherein nucleotides are equally as important.

The bioorthogonal functional moieties described herein are non-native, non-perturbing bioorthogonal chemical moieties that possess unique chemical functionality that can be modified through highly selective reactions. In particular these incorporated nucleosides are labeled using labeling reagents which comprise a chemical handle that will selectively form a covalent bond with the nucleoside in the presence of the cellular milieu.

Nucleoside analogues (or nucleotide analogues) suitable for use in the methods described herein include any nucleoside analogue (or nucleotide analogue), as defined herein, that contains a reactive bioorthoganol moiety that can undergo a [3+2] cycloaddition or Staudinger ligation. In some embodiments, the reactive bioorthoganol moiety is carried by the base of the nucleoside (or nucleotide). The base carrying the reactive bioorthoganol moiety can be a purine (e.g., adenine or guanine) or a pyrimidine (e.g., cytosine, uracil or thymine). In certain embodiments, the base is uracil; in some such embodiments, uracil carries the reactive bioorthoganol moiety on the 5-position. In certain embodiments, the base is adenine; in some such embodiments, adenine carries the reactive bioorthoganol moiety on the 5-position. In certain embodiments, the bioorthoganol moiety is indirectly attached to the base, while in other embodiments the bioorthoganol moiety is directly covalently attached to the base. Non-limiting examples of the nucleoside analogues that may be used in the methods described herein include a 5-ethynyl-2′-deoxyuracil (also termed herein ethynyluracil or EdU) which includes substituted EdU, such as 3′-fluoro-EdU, an EdC (5-ethynyl-2′-deoxycytidine, also termed herein ethynylcytosine) which includes substituted EdC, an EU (5-ethynyl uridine), an EC (5-ethynyl cytidine) and a 5-azido-2′-deoxyuracil (also termed herein azidouracil or AdU) as well as their triphosphate and phosphoramidite forms. EdU can be synthesized essentially as described by Yu and Oberdorfer, Synlett, 1:86-88 (2000), and AdU can be synthesized using a method similar to that described in Sunthankar et al., Anal. Biochem., 258:195-201 (1998) to synthesize azido-dUMP. EdU is also commercially available from Thermo Fisher Scientific (Waltham, Mass.).

In certain embodiments, the reactive bioorthoganol moiety is carried by the sugar (ribose and deoxyribose) of the nucleoside (or nucleotide). In certain embodiments, the bioorthoganol moiety is indirectly attached to the sugar, while in other embodiments the bioorthoganol moiety is directly covalently attached to the sugar. In certain embodiments, the nucleotide is a nucleotide monophosphate with the reactive bioorthogonal moiety attached to the phosphate moiety. In certain embodiments, the nucleotide is a nucleotide diphosphate with the reactive bioorthoganol moiety attached to the terminal phosphate moiety. In certain embodiments, the nucleotide is a nucleotide triphosphate with the reactive bioorthoganol moiety attached to the terminal phosphate moiety. The sugar carrying the reactive bioorthoganol moiety can be covalently attached to a purine (e.g., adenine or guanine) or a pyrimidine (e.g., cytosine, uracil or thymine). In certain embodiments, the base is uracil, while in other embodiments the base is adenine. Non-limiting examples of the nucleotide triphosphate analogues that may be used in the methods described herein include N₃-dATP (azide-dATP), N₃-dUTP (azide-dUTP), N₃-dTTP (azide-dTTP), N₃-dGTP (azide-dGTP), N₃-dCTP (azide-dCTP), E-dATP (ethynyl-dATP), E-dUTP (ethynyl-dUTP), E-dGTP (ethynyl-dGTP), E-dCTP (ethynyl-dCTP), and E-dTTP (ethynyl-dTTP), or chain terminating dideoxy compounds such as 3′-Azido-2′,3′-dideoxyadenosine, 3′-Azido-3′-deoxythymidine (AZT), 5′-Azido-5′-deoxythymidine, 5-(1-ethynyl)-2′-O-methyluridine, 5-(1-propynyl)-2′-deoxyuridine, 5-(propargyloxy)-2′-deoxyuridine, and 8-Azido-2′-deoxyadenosine.

The reactive bioorthoganol moiety can be a 1,3-dipole such as a nitrile oxide, an azide, a diazomethane, a nitrone or a nitrile imine. In certain embodiments, the 1,3-dipole is an azide. Alternatively, the reactive bioorthoganol moiety can be a dipolarophile such as an alkene (e.g., vinyl, propylenyl, and the like) or an alkyne (e.g., ethynyl, propynyl, and the like). In certain embodiments, the dipolarophile is an alkyne, such as, for example, an ethynyl group.

Chemical Modification of Nucleic Acids Containing Azide, Alkyne or Phosphine Moieties:

The nucleic acids that can be chemically modified using the methods described herein contain azide moieties, alkyne moieties or phosphine moieties that are incorporated into nucleic acids using various amplification techniques utilizing nucleobases that contain azide moieties, alkyne moieties or phosphine moieties. Such nucleobases have been chemically synthesized as described herein. These azide moieties, alkyne moieties and phosphine moieties are non-native, non-perturbing bioorthogonal chemical moieties that possess unique chemical functionality that can be modified through highly selective reactions. Non-limiting examples of such reactions used in the methods described herein are those wherein the chemical labeling of nucleic acids that contain azide moieties or alkyne moieties utilize Copper(I)-catalyzed Azide-Alkyne Cycloaddition, (CuAAC) also referred to herein as “click” chemistry, the chemical labeling of nucleic acids that contain azide moieties or phosphine moieties utilize Staudinger ligation, and the chemical labeling of nucleic acids that contain activated-alkyne moieties or activated-alkyne reactive moieties.

“Click” Chemistry:

Azides and terminal or internal alkynes can undergo a 1,3-dipolar cycloaddition (Huisgen cycloaddition) reaction to give a 1,2,3-triazole. However, this reaction requires long reaction times and elevated temperatures. Alternatively, azides and terminal alkynes can undergo Copper (I)-catalyzed Azide-Alkyne Cycloaddition (CuAAC) at room temperature. Such copper (I)-catalyzed azide-alkyne cycloadditions, also known as “click” chemistry, is a variant of the Huisgen 1,3-dipolar cycloaddition wherein organic azides and terminal alkynes react to give 1,4-regioisomers of 1,2,3-triazoles. Examples of “click” chemistry reactions are described by Sharpless et al. (U.S. Patent Application Publication No. 2005/0222427, PCT Publication No. WO 2003/101972; Lewis et al., Angewandte Chemie-Int'l Ed. 41:1053-1057 (2002); method reviewed in Kolb, et al., Angew. Chem. Intl. Ed., 40:2004-2021 (2001)), which developed reagents that react with each other in high yield and with few side reactions in a heteroatom linkage (as opposed to carbon-carbon bonds) in order to create libraries of chemical compounds. As described herein, “click” chemistry is used in the methods for labeling nucleic acids.

The copper used as a catalyst for the “click chemistry reaction used in the methods described herein to conjugate a label (reporter molecule, solid support or carrier molecule) to a nucleic acid is in the Cu(I) reduction state. The sources of Cu(I) used in such copper (I)-catalyzed azide-alkyne cycloaddition can be any cuprous salt including, but not limited to, cuprous halides such as cuprous bromide or cuprous iodide. However, this regioselective cycloaddition can also be conducted in the presence of a metal catalyst and a reducing agent. In certain embodiments, copper can be provided in the Cu(II) reduction state (for example, as a salt, such as but not limited to Cu(NO₃)₂, Cu(OAc)₂ or CuSO₄), in the presence of a reducing agent wherein Cu(I) is formed in situ by the reduction of Cu(II). Such reducing agents include, but are not limited to, ascorbate, Tris(2-Carboxyethyl) Phosphine (TCEP), 2,4,6-trichlorophenol (TCP), NADH, NADPH, thiosulfate, metallic copper, quinone, hydroquinone, vitamin K1, glutathione, cysteine, 2-mercaptoethanol, dithiothreitol, Fe²⁺, Co²⁺, or an applied electric potential. In other embodiments, the reducing agents include metals selected from Al, Be, Co, Cr, Fe, Mg, Mn, Ni, Zn, Au, Ag, Hg, Cd, Zr, Ru, Fe, Co, Pt, Pd, Ni, Rh, and W.

The copper (I)-catalyzed azide-alkyne cycloadditions for labeling nucleic acids can be performed in water and a variety of solvents, including mixtures of water and a variety of (partially) miscible organic solvents including alcohols, dimethyl sulfoxide (DMSO), dimethyl formamide (DMF), tert-butanol (tBuOH) and acetone.

Without limitation to any particular mechanism, copper in the Cu(I) state is a preferred catalyst for the copper (I)-catalyzed azide-alkyne cycloaddition, or “click” chemistry reactions, used in the methods described herein. Certain metal ions are unstable in aqueous solvents, by way of example Cu(I), therefore stabilizing ligands/chelators can be used to improve the reaction. In certain embodiments at least one copper chelator is used in the methods described herein, wherein such chelators bind copper in the Cu(I) state. In certain embodiments at least one copper chelator is used in the methods described herein, wherein such chelators bind copper in the Cu(II) state. In certain embodiments, the Cu(I) chelator is a 1,10 phenanthroline-containing Cu(I) chelator. Non-limiting examples of such phenanthroline-containing Cu(I) chelators include, but are not limited to, bathophenanthroline disulfonic acid (4,7-diphenyl-1,10-phenanthroline disulfonic acid) and bathocuproine disulfonic acid (BCS; 2,9-dimethyl-4,7-diphenyl-1,10-phenanthroline disulfonate). Other chelators used in such methods include, but are not limited to, N-(2-acetamido) iminodiacetic acid (ADA), pyridine-2,6-dicarboxylic acid (PDA), S-carboxymethyl-L-cysteine (SCMC), trientine, tetraehylenepolyamine (TEPA), NNNN-tetrakis(2-pyridylmethyl)ethylenediamine (TPEN), EDTA, neocuproine, N-(2-acetamido)iminodiacetic acid (ADA), pyridine-2,6-dicarboxylic acid (PDA), S-carboxymethyl-L-cysteine (SCMC), tris-(benzyl-triazolylmethyl)amine (TBTA), or a derivative thereof. Most metal chelators, a wide variety of which are known in the chemical, biochemical, and medical arts, are known to chelate several metals, and thus metal chelators in general can be tested for their function in 1,3 cycloaddition reactions catalyzed by copper. In certain embodiments, histidine is used as a chelator, while in other embodiments glutathione is used as a chelator and a reducing agent.

The concentration of the reducing agents used in the “click” chemistry reaction described herein can be in the micromolar to millimolar range. In certain embodiments the concentration of the reducing agent is from about 100 micromolar to about 100 millimolar. In other embodiments the concentration of the reducing agent is from about 10 micromolar to about 10 millimolar. In other embodiments the concentration of the reducing agent is from about 1 micromolar to about 1 millimolar.

In certain embodiments of the methods described herein for labeling nucleic acids using “click” chemistry, at least one copper chelator is added after Cu(II) used in the reaction has been contacted with a reducing agent. In other embodiments, at least one copper chelator can be added immediately after contacting Cu(II) with a reducing agent. In other embodiments, the copper chelator(s) is added between about five seconds and about twenty-four hours after Cu(II) and a reducing agent have been combined in a reaction mixture. In other embodiments, at least one copper chelator can be added any time to a reaction mixture that includes Cu(II) and a reducing agent, such as, by way of example only, immediately after contacting Cu(II) and a reducing agent, or within about five minutes of contacting Cu(II) and a reducing agent in the reaction mixture. In some embodiments, at least one copper chelator can be added between about five seconds and about one hour, between about one minute and about thirty minutes, between about five minutes and about one hour, between about thirty minutes and about two hours, between about one hour and about twenty-four hours, between about one hour and about five hours, between about two hours and about eight hours, after Cu(II) and a reducing agent have been combined for use in a reaction mixture.

In other embodiments, one or more copper chelators can be added more than once to such “click” chemistry reactions. In embodiments in which more than one copper chelator is added to a reaction, two or more of the copper chelators can bind copper in the Cu(I) state or, one or more of the copper chelators can bind copper in the Cu(I) state and one or more additional chelators can bind copper in the Cu(II) state. In certain embodiments, one or more copper chelators can be added after the initial addition of a copper chelator to the “click” chemistry reaction. In certain embodiments, the one or more copper chelators added after the initial addition of a copper chelator to the reaction can be the same or different from a copper chelator added at an earlier time to the reaction.

The concentration of a copper chelator used in the “click” chemistry reaction described herein can be determined and optimized using methods well known in the art, including those disclosed herein using “click” chemistry to label nucleic acids followed by detecting such labeled nucleic acids to determine the efficiency of the labeling reaction and the integrity of the labeled nucleic acid(s). In certain embodiments, the chelator concentrations used in the methods described herein is in the micromolar to millimolar range, by way of example only, from 1 micromolar to 100 millimolar. In certain embodiments the chelator concentration is from about 10 micromolar to about 10 millimolar. In other embodiments the chelator concentration is from about 50 micromolar to about 10 millimolar. In other embodiments the chelator, can be provided in a solution that includes a water miscible solvent such as, alcohols, dimethyl sulfoxide (DMSO), dimethyl formamide (DMF), tert-butanol (tBuOH) and acetone. In other embodiments the chelator, can be provided in a solution that includes a solvent such as, for example, dimethyl sulfoxide (DMSO) or dimethylformamide (DMF).

In certain embodiments of the methods for labeling nucleic acids utilizing “click” chemistry described herein, the nucleic acid can possess an azide moiety, whereupon the label possesses an alkyne moiety, whereas in other embodiments the nucleic acid can possess an alkyne moiety, and the label possesses an azide moiety.

Staudinger Ligation:

The Staudinger reaction, which involves reaction between trivalent phosphorous compounds and organic azides (Staudinger et al., Helv. Chim. Acta, 2:635 (1919)), has been used for a multitude of applications. (Gololobov et al., Tetrahedron, 37: 437 (1980)); (Gololobov et al., Tetrahedron, 48: 1353 (1992)). There are almost no restrictions on the nature of the two reactants. The Staudinger ligation is a modification of the Staudinger reaction in which an electrophilic trap (usually a methyl ester) is placed on a triaryl phosphine. In the Staudinger ligation, the aza-ylide intermediate rearranges, in aqueous media, to produce an amide linkage and the phosphine oxide, ligating the two molecules together, whereas in the Staudinger reaction the two products are not covalently linked after hydrolysis. Such ligations have been described in U.S. Patent Application Publication No. 2006/0276658. In certain embodiments, the phosphine can have a neighboring acyl group such as an ester, thioester or N-acyl imidazole (i.e. a phosphinoester, phosphinothioester, phosphinoimidazole) to trap the aza-ylide intermediate and form a stable amide bond upon hydrolysis. In certain embodiments, the phosphine can be a di- or triarylphosphine to stabilize the phosphine. The phosphines used in the Staudinger ligation methods described herein to conjugate a label to a nucleic acid include, but are not limited to, cyclic or acyclic, halogenated, bisphosphorus, or even polymeric. Similarly, the azides can be alkyl, aryl, acyl or phosphoryl. In certain embodiments, such ligations are carried out under oxygen-free anhydrous conditions.

In certain embodiments of the methods for labeling nucleic acid utilizing Staudinger ligation described herein, the nucleic acid can possess an azide moiety, whereupon the label possesses a phosphine moiety, whereas in other embodiments the nucleic acid can possess a phosphine moiety, and the label possesses an azide moiety.

Activated-Alkyne Chemistry:

Azides and alkynes can undergo catalyst-free [3+2] cycloaddition by a using the reaction of activated alkynes with azides. Such catalyst free [3+2] cycloaddition can be used in methods described herein to conjugate a label (reporter molecule, solid support or carrier molecule) to a nucleic acid. Alkynes can be activated by ring strain such as, by way of example only, eight membered ring structures, appending electron-withdrawing groups to such alkyne rings, or alkynes can be activated by the addition of a Lewis acid such as, by way of example only, Au(I) or Au(III).

In certain embodiments of the methods for labeling nucleic acids utilizing activated alkynes described herein, the nucleic acid can possess an azide moiety, whereupon the label possesses an activated alkyne moiety, whereas in other embodiments the nucleic acid can possess an activated alkyne moiety, and the label possesses an azide moiety.

After nucleic acids have been modified with azide moieties, alkyne moieties or phosphine moieties, they can be reacted under appropriate conditions to form conjugates with reporter molecules, solid supports or carrier molecules. In the methods and compositions described herein the azide moiety, alkyne moiety or phosphine moiety is used as a reactive functional group or chemical handle on the modified nucleic acid wherein an azide reactive moiety on a label, or an alkyne reactive moiety on a label, or a phosphine reactive moiety on a label is reacted with the modified nucleic acid to form a covalent conjugate comprising the nucleic acid and at least one label.

The methods as described herein that utilize cycloaddition reactions to label nucleic acids can be carried out at room temperature in aqueous conditions with excellent regioselectivity by the addition of catalytic amounts of Cu(I) salts to the reaction mixture. See, e.g., Tomoe, et al., Org. Chem. 67:3057-3064 (2002) and, Rostovtsev, et al., Angew. Chem. Int. Ed., 41:2596-2599 (2002). The resulting five-membered ring resulting from “click” chemistry cycloaddition is not generally reversible in reducing environments and is stable against hydrolysis for extended periods in aqueous environments. Thus, nucleic acids attached to a labeling agent via such five-membered ring are stable.

The labels used in the methods and compositions described herein, can contain at least one alkyne moiety or at least one phosphine moiety capable of reacting with an azide moiety. The labels used in the methods and compositions described herein, can contain at least one azide moiety capable of reacting with an alkyne moiety or a phosphine moiety. The labels used in the methods and compositions described herein, can contain at least one phosphine moiety capable of reacting with an azide moiety. In certain embodiments, the phosphine moieties of the labels described herein are triarylphosphine moieties.

Labeling Reagents and Methods of Use:

In general, for ease of understanding the present disclosure, the components for colorimetric labeling of nucleic acids through the incorporation of nucleoside or nucleotide analogs will first be described in detail, followed by a description of the colorimetric labeling methods. This will be followed by some embodiments in which such labeled nucleic acid is used to measure cell proliferation. Exemplified methods are then disclosed.

Certain embodiments of the present disclosure provide an adapter linker having structural formula (I):

Az-L-TM (I)

wherein, Az is an azide moiety, L is a spacer, and TM is a tetrazine functional group. In certain embodiments, the spacer comprises 1 to 20 PEG groups. In certain embodiments, the spacer comprises 2 to 10 PEG groups. In certain embodiments, the spacer comprises 4 PEG groups. In certain embodiments, the spacer comprises 2 PEG groups.

1. “Double Click Reaction”

Certain embodiments of the present disclosure provide methods of labeling a nucleic acid polymer, the methods comprising:
a) incubating a sample with an effective amount of an alkynyl-modified nucleoside analogue, thereby forming an alkynyl-modified nucleic acid polymer;
b) incubating the alkynyl-modified nucleic acid polymer with an adapter linker of structural formula (I) under conditions such that the alkynyl moiety of the alkynyl-modified nucleic acid polymer forms a covalent link with the azide moiety of the adapter linker, thereby forming an adapter intermediate; and
c) incubating the adapter intermediate with a detectable label comprising a cycloalkene group under conditions such that a covalent link forms between the tetrazine functional group of the adapter intermediate and the cycloalkene group of the detectable label, thereby forming a labeled nucleic acid polymer.

In certain embodiments, the alkynyl-modified nucleoside analogue is an EdU or an EdC. In certain embodiments, the cycloalkene group is a trans-cycloalkene or a cyclopropene. In certain embodiments, the detectable label is a colorimetric label. In certain embodiments, the colorimetric label is horseradish peroxidase, alkaline phosphatase, beta-galactosidase, glucose oxidase or beta-lactamase. In certain embodiments, the horseradish peroxidase is a trans-cyclooctene-horseradish peroxidase conjugate.

In certain embodiments, the step of contacting the alkynyl-modified nucleic acid polymer with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the alkynyl-modified nucleic acid polymer with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the alkynyl-modified nucleic acid polymer with the adapter linker is performed in the presence of a copper chelator.

2. “Fluorescent Intermediate Method”:

In certain embodiments of the present disclosure, methods for labeling nucleic acid polymers are provided, the methods comprising:
a) contacting a nucleic acid polymer with an effective amount of an alkynyl-modified nucleoside analogue, thereby forming an alkynyl-modified nucleic acid polymer;
b) contacting the alkynyl-modified nucleic acid polymer with an azide-modified fluorescent dye under conditions such that the alkynyl moiety of the alkynyl-modified nucleoside analogue forms a covalent link with the azide moiety of the fluorescent dye, thereby forming a fluorescent intermediate;
c) contacting the fluorescent intermediate with an anti-fluorescent dye antibody that binds to the azide-modified fluorescent dye, thereby forming an antibody-bound intermediate; and
d) contacting the cell with a secondary antibody conjugated to a detectable label, wherein the secondary antibody binds to the anti-fluorescent dye antibody, thereby forming a labeled nucleic acid polymer.

In certain embodiments of the present disclosure, methods for labeling nucleic acid polymers are provided, the methods comprising:
a) contacting a nucleic acid polymer with an effective amount of an alkynyl-modified nucleoside analogue, thereby forming an alkynyl-modified nucleic acid polymer;
b) contacting the alkynyl-modified nucleic acid polymer with an azide-modified fluorescent dye under conditions such that the alkynyl moiety of the alkynyl-modified nucleoside analogue forms a covalent link with the azide moiety of the fluorescent dye, thereby forming a fluorescent intermediate; and
c) contacting the cell with an anti-fluorescent dye antibody conjugated to a detectable label, wherein the antibody binds the azide-modified fluorescent dye, thereby forming a labeled nucleic acid polymer.

In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the alkynyl-modified nucleic acid polymer with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the alkynyl-modified nucleic acid polymer with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the alkynyl-modified nucleic acid polymer with the azide-modified fluorescent dye is performed in the presence of a copper chelator.

3. “Biotin Intermediate Method”:

In certain embodiments of the present disclosure, methods for labeling nucleic acid polymers are provided, the methods comprising:
a) contacting a nucleic acid polymer with an effective amount of an alkynyl-modified nucleoside analogue, thereby forming an alkyne-modified nucleic acid polymer;
b) contacting the alkyne-modified nucleic acid polymer with an azide-modified biotin under conditions such that the alkynyl moiety of the alkynyl-modified nucleoside analogue forms a covalent link with the azide moiety of the biotin, thereby forming a biotin-modified intermediate; and
c) contacting the biotin-modified intermediate with a (strept)avidin conjugated to a detectable label, thereby forming a labeled nucleic acid polymer.

In certain embodiments, the alkynyl-modified nucleoside analogue is an EdU or an EdC. In certain embodiments, the detectable label is a colorimetric label. In certain embodiments, the colorimetric label is selected from horseradish peroxidase, alkaline phosphatase, beta-galactosidase, glucose oxidase and beta-lactamase.

In certain embodiments, the step of contacting the alkynyl-modified nucleic acid polymer with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the alkynyl-modified nucleic acid polymer with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the alkynyl-modified nucleic acid polymer with the azide-modified biotin is performed in the presence of a copper chelator.

Labeling of Nucleic Acid Polymers in Cells:

As described herein, some embodiments of the present disclosure relate to incorporation of labels into nucleic acid polymers in cells in culture. In certain embodiments, the cells are grown in standard tissue culture plastic ware. Such cells include normal and transformed cells derived. In certain embodiments, the cells are of mammalian (e.g., human or animal, such as rodent or simian) origin. Mammalian cells may be of any fluid, organ or tissue origin (e.g., blood, brain, liver, lung, heart, bone, and the like) and of any cell types (e.g., basal cells, epithelial cells, platelets, lymphocytes, T -cells, B-cells, natural killer cells, macrophages, tumor cells, and the like).

Cells suitable for use in the methods of the present disclosure may be primary cells, secondary cells or immortalized cells (i.e., established cell lines). They may have been prepared by techniques well-known in the art (for example, cells may be obtained by drawing blood from a patient or a healthy donor) or purchased from immunological and microbiological commercial resources (for example, from the American Type Culture Collection, Manassas, Va.). Alternatively or additionally, cells may be genetically engineered to contain, for example, a gene of interest such as a gene expressing a growth factor or a receptor.

Cells to be used in the methods disclosed herein may be cultured according to standard culture techniques. For example, cells are often grown in a suitable vessel in a sterile environment at 37° C. in an incubator containing a humidified 95% air/5% CO₂ atmosphere. Vessels may contain stirred or stationary cultures. Various cell culture media may be used including media containing undefined biological fluids such as fetal calf serum. Cell culture techniques are well known in the art, and established protocols are available for the culture of diverse cell types (see, for example, R. I. Freshney, “Culture of Animal Cells: A Manual of Basic Technique”, 2nd Edition, 1987, Alan R. Liss, Inc.).

Incorporation of nucleoside analogues into DNA by DNA replication is a process well-known in the art. In general, nucleoside analogues are transported across the cell membrane by nucleoside transporters and are phosphorylated in cells by kinases to their triphosphate forms. The nucleoside analogue triphosphates then compete with the naturally-occurring deoxyribonucleotides as substrates of cellular DNA polymerases. Such a process is used for the incorporation of ³H-thymidine and 5′-bromo-2′deoxyuridine (BrdU) into DNA for labeling purposes as well as in cancer therapy (D. Kufe et al., Blood, 64:54-58 (1984); Beutler, Lancet, 340:952-956 (1992); Hui and Reitz, Am. J. Health-Syst. Pharm., 54:162-170 (1997); Iwasaki et al., Blood, 90:270-278 (1997)) and in the treatment of human immunodeficiency virus infection (Balzarini, Pharm. World Sci., 16: 113-126 (1994)).

Contacting the cells in vitro with an effective amount of a nucleoside analogue such that the nucleoside analogue is incorporated into DNA of the cell may be carried out using any suitable protocol. In certain preferred embodiments, the nucleoside analogue is incorporated into DNA using exponentially growing cells or cells in the S-phase of the cell cycle (i.e., the synthesis phase). If desired, cells may be synchronized in early S-phase by serum deprivation before the labeling-pulse procedure.

The step of contacting a cell with an effective amount of a nucleoside analogue may be performed, for example, by incubating the cell with the nucleoside analogue under suitable incubation conditions (e.g., in culture medium at 37° C.). In certain situations, it may be desirable to avoid disturbing the cells in any way (e.g., by centrifugation steps or temperature changes) that may perturb their normal cell cycling patterns. The incubation time will be dependent on the cell population's rate of cell cycling entry and progression. Optimization of incubation time and conditions is within the skill in the art.

Following incorporation of the nucleotide analogue into the DNA of in vitro cells, the step of contacting the cells with a staining reagent comprising a bioorthogonal moiety and a label, for example an adapter linker of structural formula (I), an azide-modified fluorescent dye, or an azide-modified biotin, may be performed by any suitable method. In some embodiments, the cells are incubated in the presence of the staining reagent in a suitable incubation medium (e.g., culture medium) at 37° C. and for a time sufficient for the reagent to penetrate into the cell and react with any nucleotide analogue incorporated into the DNA of the cells. Optimization of the concentration of staining reagent, cycloaddition reaction time and conditions is within the skill in the art.

As already described above, in embodiments where the presence of exogenous Cu(I) is not desirable, the [3+2] cycloaddition may be carried out using a staining reagent that comprises the second reactive unsaturated group, a label, and a Cu(I) chelating moiety.

In embodiments where the staining reagent does not exhibit high cell permeability, permeabilization may be performed to facilitate access of the staining reagent to cellular cytoplasm, or intracellular components or structures of the cells. In particular, permeabilization may allow a reagent to enter into a cell and reach a concentration within the cell that is greater than that which would normally penetrate into the cell in the absence of such permeabilization treatment.

Permeabilization of the cells may be performed by any suitable method (see, for example, Goncalves et al., Neurochem. Res. 25:885-894 (2000)). Such methods include, but are not limited to, exposure to a detergent (such as CHAPS, cholic acid, deoxycholic acid, digitonin, n-dodecyl-13-D-maltoside, lauryl sulfate, glycodeoxycholic acid, n-lauroylsarcosine, saponin, and Triton X-100) or to an organic alcohol (such as methanol and ethanol). Other permeabilization methods comprise the use of certain peptides or toxins that render membranes permeable (see, for example, Aguilera et al., FEBS Lett., 462: 273-277 (1999); Bussing et al., Cytometry, 37:133-139 (1999)). Selection of an appropriate permeabilizing agent and optimization of the incubation conditions and time can easily be performed by one of ordinary skill in the art.

Labeling of Nucleic Acid Polymers in Tissues or Organisms:

As described herein, the present disclosure also provides methods for labeling nucleic acid polymers in organisms (i.e., living biological systems). Unless otherwise stated, the reagents and [3+2] cycloaddition conditions used in these methods are analogous to those described above for the methods of labeling nucleic acid polymers in cells and can easily be determined and/or optimized by one skilled in the art.

These methods can be performed using techniques and procedures as described herein for methods of labeling nucleic acid polymers in cells and organisms. With such methods, the manner of performing the contacting and/or administering steps, type of reagents (i.e., with or without a copper chelating moiety), type of label, and techniques for the detection of such labels are analogous to those described for other methods provided herein relating to labeling nucleic acid polymers in cells or in organisms.

Methods of labeling of the present disclosure may be performed using any living system that has or can develop the ability to act or function independently. Thus labeling methods of the present disclosure may be performed in unicellular or multicellular systems, including, humans, animals, plants, bacteria, protozoa, and fungi. In certain preferred embodiments, the labeling methods provided herein are performed in a human or another mammal (e.g., mouse, rat, rabbit, dog, cat, cattle, swine, sheep, horse or primate).

Administration of a nucleoside analogue to an organism may be performed using any suitable method that results in incorporation of the nucleoside analogue into the DNA of cells of the organism.

For example, the nucleoside analogue may be formulated in accordance with conventional methods in the art using a physiologically and clinically acceptable solution. Proper solution is dependent upon the route of administration chosen. Suitable routes of administration can, for example, include oral, rectal, transmucosal, transcutaneous, or intestinal administration; parenteral delivery, including intramuscular, subcutaneous, intramedullary injections, as well as intrathecal, direct intraventricular, intravenous, intraperitoneal, intranasal, or intraocular injections. Alternatively, the nucleoside analogue preparation can be administered in a local rather than systemic manner, for example, via injection directly into a specific tissue, often in a depot or sustained release formulation.

Following incorporation of the nucleoside analogue into the DNA of cells of the organism, the step of contacting at least one cell of the organism with a reagent comprising a bioorthogonal moiety attached to a label may be performed by any suitable method that allows for the [3+2] cycloaddition to take place.

In certain embodiments, cells are collected (e.g., by drawing blood from the organism), isolated from a tissue obtained by biopsy (e.g., needle biopsy, laser capture micro dissection or incisional biopsy) or isolated from an organ or part of an organ (e.g., harvested at autopsy). The cells can then be submitted to the [3+2] cycloaddition staining as described above.

In other embodiments, a tissue obtained by biopsy or an organ or part of an organ harvested at autopsy may be prepared for staining as known in the art (e.g., fixed, embedded in paraffin and sectioned) and incubated in the presence of the [3+2] cycloaddition reagent (e.g., after de-waxing).

Cellular Proliferation Assays:

A. Methods for Measuring Cellular Proliferation:

As described herein, methods for measuring cellular proliferation or cellular proliferation rates according to the present disclosure may be used in a wide variety of applications, including, but not limited to characterization of cell lines, optimization of cell culture conditions, characterization of cellular proliferation in normal, diseased and injured tissues, and diagnosis of a variety of diseases and disorders in which cellular proliferation is involved.

1. “Double Click Reaction”:

According to certain embodiments, the present disclosure provides methods of measuring cellular proliferation, the methods comprising:
a) contacting a cell with an effective amount of an alkynyl-modified nucleoside analogue, such that the alkynyl-modified nucleoside analogue is incorporated into DNA of the cell;
b) contacting the cell with an adapter linker of structural formula (I) under conditions such that the alkynyl moiety of the alkynyl-modified nucleoside analogue forms a covalent link with the azide moiety of the adapter linker, thereby forming an adapter intermediate;
c) contacting the cell with a detectable label comprising a cycloalkene group under conditions such that a covalent link forms between the tetrazine functional group of the adapter intermediate and the cycloalkene group of the detectable label; and
d) measuring the amount of detectable label incorporated into the DNA, wherein the amount of label indicates the extent of cellular proliferation.

In certain embodiments of the present disclosure, methods of measuring cellular proliferation in an organism are provided, the methods comprising:
a) administering to an organism an effective amount of an alkynyl-modified nucleoside analogue, such that the alkynyl-modified nucleoside analogue is incorporated into DNA of cells of the organism;
b) contacting at least one cell of the organism with an adapter linker of structural formula (I) under conditions such that the alkynyl moiety of the alkynyl-modified nucleoside analogue forms a covalent link with the azide moiety of the adapter linker, thereby forming an adapter intermediate;
c) contacting the at least one cell of the organism with a detectable label comprising a cycloalkene group under conditions such that a covalent link forms between the tetrazine functional group of the adapter intermediate and the cycloalkene group of the detectable label; and
d) measuring the amount of detectable label incorporated into the DNA, wherein the amount of label indicates the extent of cellular proliferation.

In certain embodiments, the alkynyl-modified nucleoside analogue is an EdU or an EdC. In certain embodiments, the cycloalkene group is a trans-cycloalkene or a cyclopropene. In certain embodiments, the detectable label is a colorimetric label. In certain embodiments, the colorimetric label is selected from horseradish peroxidase, alkaline phosphatase, beta-galactosidase, glucose oxidase or beta-lactamase. In certain embodiments, the horseradish peroxidase is a trans-cyclooctene-horseradish peroxidase conjugate. In certain embodiments, the cell is in a multi-well plate.

In certain embodiments, the step of contacting the cell with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the adapter linker is performed in the presence of a copper chelator.

2. “Fluorescent Intermediate Reaction”:

According to certain embodiments of the present disclosure, methods for measuring cellular proliferation are provided, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody that binds to the azide-modified fluorescent dye;
d) contacting the cell with a secondary antibody conjugated to a detectable label, wherein the secondary antibody binds to the anti-fluorescent dye antibody; and
e) measuring the amount of detectable label, wherein the amount of label indicates the extent of cellular proliferation.

In certain embodiments, methods are provided for measuring cellular proliferation, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody conjugated to a detectable label, wherein the antibody binds to the azide-modified fluorescent dye; and
d) measuring the amount of detectable label, wherein the amount of label indicates the extent of cellular proliferation.

According to certain embodiments of the present disclosure, methods for measuring cellular proliferation in an organism are provided, the methods comprising:
a) administering to an organism an effective amount of an alkynyl-modified nucleoside analogue, such that the alkynyl-modified nucleoside analogue is incorporated into DNA of cells of the organism;
b) contacting at least one cell of the organism with an azide-modified fluorescent dye under conditions such that the alkynyl moiety of the alkynyl-modified nucleoside analogue forms a covalent link with the azide moiety of the fluorescent dye;
c) contacting the at least one cell of the organism with an anti-fluorescent dye antibody that binds to the azide-modified fluorescent dye;
d) contacting the at least one cell of the organism with a secondary antibody conjugated to a detectable label, wherein the secondary antibody binds to the anti-fluorescent dye antibody; and
e) measuring the amount of detectable label, wherein the amount of label indicates the extent of cellular proliferation.

In certain embodiments, methods are provided for measuring cellular proliferation in an organism, the methods comprising:
a) administering to an organism an effective amount of an alkynyl-modified nucleoside analogue, such that the alkynyl-modified nucleoside analogue is incorporated into DNA of cells of the organism;
b) contacting at least one cell of the organism with an azide-modified fluorescent dye under conditions such that the alkynyl moiety of the alkynyl-modified nucleoside analogue forms a covalent link with the azide moiety of the fluorescent dye;
c) contacting the at least one cell of the organism with an anti-fluorescent dye antibody conjugated to a detectable label, wherein the antibody binds to the azide-modified fluorescent dye; and
d) measuring the amount of detectable label, wherein the amount of label indicates the extent of cellular proliferation.

In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the cell with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified fluorescent dye is performed in the presence of a copper chelator.

3. “Biotin Intermediate Method”:

According to certain embodiments of the present disclosure, methods of measuring cellular proliferation are provided, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified biotin under conditions such that the alkynyl moiety of the alkynyl-modified nucleoside analogue forms a covalent link with the azide moiety of the biotin;
c) contacting the cell with a (strept)avidin conjugated to a detectable label; and
d) measuring the amount of detectable label, wherein the amount of label indicates the extent of cellular proliferation.

According to certain embodiments of the present disclosure, methods of measuring cellular proliferation in an organism are provided, the methods comprising:
a) administering to an organism an effective amount of an alkynyl-modified nucleoside analogue, such that the alkynyl-modified nucleoside analogue is incorporated into DNA of cells of the organism;
b) contacting at least one cell of the organism with an azide-modified biotin under conditions such that the alkynyl moiety of the alkynyl-modified nucleoside analogue forms a covalent link with the azide moiety of the biotin;
c) contacting the at least one cell of the organism with a (strept)avidin conjugated to a detectable label; and
d) measuring the amount of detectable label, wherein the amount of label indicates the extent of cellular proliferation.

In certain embodiments, the alkynyl-modified nucleoside analogue is an EdU or an EdC. In certain embodiments, the detectable label is a colorimetric label. In certain embodiments, the colorimetric label is selected from horseradish peroxidase, alkaline phosphatase, beta-galactosidase, glucose oxidase and beta-lactamase.

In certain embodiments, the step of contacting the cell with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified biotin is performed in the presence of a copper chelator.

B. Methods for Measuring Cellular DNA Synthesis:

In one aspect is provided a method for measuring cellular DNA synthesis or a change in cellular DNA synthesis, which can be measured as cell proliferation.

1. “Double Click Reaction”:

According to certain embodiments of the present disclosure, methods of measuring cellular DNA synthesis are provided, the methods comprising:
a) contacting a cell with an effective amount of an alkynyl-modified nucleoside analogue, such that the alkynyl-modified nucleoside analogue is incorporated into DNA of the cell;
b) contacting the cell with an adapter linker of structural formula (I) under conditions such that the alkynyl moiety of the alkynyl-modified nucleoside analogue forms a covalent link with the azide moiety of the adapter linker, thereby forming an adapter intermediate;
c) contacting the cell with a detectable label comprising a cycloalkene group under conditions such that a covalent link forms between the tetrazine functional group of the adapter intermediate and the cycloalkene group of the detectable label; and
d) measuring the amount of detectable label incorporated into the DNA, wherein the amount of label indicates the extent of cellular DNA synthesis.

In certain embodiments, the methods measure a change in cellular DNA synthesis. In certain embodiments, the alkynyl-modified nucleoside analogue is an EdU or an EdC. In certain embodiments, the cycloalkene group is a trans-cyclooctene or a cyclopentene. In certain embodiments, the detectable label is a colorimetric label. In certain embodiments, the colorimetric label is selected from horseradish peroxidase, alkaline phosphatase, beta-galactosidase, glucose oxidase or beta-lactamase. In certain embodiments, the horseradish peroxidase is a trans-cyclooctene-horseradish peroxidase conjugate. In certain embodiments, the cell is in a multi-well plate.

In certain embodiments, the step of contacting the cell with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the adapter linker is performed in the presence of a copper chelator.

2. “Fluorescent Intermediate Reaction”:

According to certain embodiments of the present disclosure, methods for measuring cellular DNA synthesis are provided, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody that binds to the azide-modified fluorescent dye;
d) contacting the cell with a secondary antibody conjugated to a detectable label, wherein the secondary antibody binds to the anti-fluorescent dye antibody; and
e) measuring the amount of detectable label, wherein the amount of label indicates the extent of DNA synthesis.

In certain embodiments provided herein, methods for measuring cellular DNA synthesis are provided, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody conjugated to a detectable label, wherein the antibody binds to the azide-modified fluorescent dye; and
d) measuring the amount of detectable label, wherein the amount of label indicates the extent of DNA synthesis.

In certain embodiments, the methods measure a change in cellular DNA synthesis. In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the cell with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified fluorescent dye is performed in the presence of a copper chelator.

3. “Biotin Intermediate Method”:

According to certain embodiments of the present disclosure, methods of measuring cellular DNA synthesis are provided, the methods comprising:
a) contacting a cell with an effective amount of an alkynyl-modified nucleoside analogue, such that the alkynyl-modified nucleoside analogue is incorporated into DNA of the cell;
b) contacting the cell with an azide-modified biotin under conditions such that the alkynyl moiety of the alkynyl-modified nucleoside analogue forms a covalent link with the azide moiety of the biotin;
c) contacting the cell with a (strept)avidin conjugated to a detectable label; and
d) measuring the amount of detectable label, wherein the amount of label indicates the extent of in DNA synthesis.

In certain embodiments, the methods measure a change in cellular DNA synthesis. In certain embodiments, the alkynyl-modified nucleoside analogue is an EdU or an EdC. In certain embodiments, the detectable label is a colorimetric label. In certain embodiments, the colorimetric label is selected from horseradish peroxidase, alkaline phosphatase, beta-galactosidase, glucose oxidase and beta-lactamase.

In certain embodiments, the step of contacting the cell with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified biotin is performed in the presence of a copper chelator.

C. Methods for Measuring Cellular RNA Synthesis:

In another aspect is provided a method for measuring cellular RNA synthesis or a change in cellular RNA synthesis, which can be measured as gene expression.

1. “Double Click Reaction”:

In certain embodiments, the present disclosure provides for methods of measuring cellular RNA synthesis, the methods comprising:
a) contacting a cell with an effective amount of an alkynyl-modified nucleoside analogue, such that the alkynyl-modified nucleoside analogue is incorporated into RNA of the cell;
b) contacting the cell with an adapter linker of structural formula (I) under conditions such that the alkynyl moiety of the alkynyl-modified nucleoside analogue forms a covalent link with the azide moiety of the adapter linker, thereby forming an adapter intermediate;
c) contacting the cell with a detectable label comprising a cycloalkene group under conditions such that a covalent link forms between the tetrazine moiety of the adapter intermediate and the cycloalkene group of the detectable label; and
d) measuring the amount of detectable label incorporated into the RNA, wherein the amount of label indicates the extent of cellular RNA synthesis.

In certain embodiments, the methods measure a change in cellular RNA synthesis. In certain embodiments, the alkynyl-modified nucleoside analogue is an EU or an EC. In certain embodiments, the cycloalkene group is a trans-cycloalkene or a cyclopropene. In certain embodiments, the detectable label is a colorimetric label. In certain embodiments, the colorimetric label is selected from horseradish peroxidase, alkaline phosphatase, beta-galactosidase, glucose oxidase or beta-lactamase. In certain embodiments, the horseradish peroxidase is a trans-cyclooctene-horseradish peroxidase conjugate. In certain embodiments, the cell is in a multi-well plate.

In certain embodiments, the step of contacting the cell with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the adapter linker is performed in the presence of a copper chelator.

2. “Fluorescent Intermediate Method”:

In certain embodiments provided herein, methods of measuring cellular RNA synthesis are provided, the methods comprising:
a) contacting a cell with an effective amount of an alkynyl-modified nucleoside analogue, such that the alkynyl-modified nucleoside analogue is incorporated into RNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody that binds to the azide-modified fluorescent dye;
d) contacting the cell with a secondary antibody conjugated to a detectable label, wherein the secondary antibody binds to the anti-fluorescent dye antibody; and
e) measuring the amount of detectable label, wherein the amount of label indicates the extent of cellular RNA synthesis.

In certain embodiments provided herein, methods of measuring cellular RNA synthesis are provided, the methods comprising:
a) contacting a cell with an effective amount of an alkynyl-modified nucleoside analogue, such that the alkynyl-modified nucleoside analogue is incorporated into RNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody conjugated to a detectable label, wherein the antibody binds to the azide-modified fluorescent dye; and
d) measuring the amount of detectable label, wherein the amount of label indicates the extent of cellular RNA synthesis.

In certain embodiments, the methods measure a change in cellular RNA synthesis. In certain embodiments, the alkynyl-modified nucleoside analogue is an EU or an EC. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the cell with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified fluorescent dye is performed in the presence of a copper chelator.

3. “Biotin Intermediate Method”:

According to certain embodiments of the present disclosure, methods of measuring cellular RNA synthesis are provided, the methods comprising:
a) contacting a cell with an effective amount of an alkynyl-modified nucleoside analogue, such that the alkynyl-modified nucleoside analogue is incorporated into RNA of the cell;
b) contacting the cell with an azide-modified biotin under conditions such that the alkynyl moiety of the alkynyl-modified nucleoside analogue forms a covalent link with the azide moiety of the biotin;
c) contacting the cell with a (strept)avidin conjugated to a detectable label; and
d) measuring the amount of detectable label, wherein the amount of label indicates the extent of cellular RNA synthesis.

In certain embodiments, the methods measure a change in cellular RNA synthesis. In certain embodiments, the alkynyl-modified nucleoside analogue is an EU or an EC. In certain embodiments, the detectable label is a colorimetric label. In certain embodiments, the colorimetric label is selected from horseradish peroxidase, alkaline phosphatase, beta-galactosidase, glucose oxidase and beta-lactamase.

In certain embodiments, the step of contacting the cell with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified biotin is performed in the presence of a copper chelator.

D. Methods for Screening Agents for Effects on Cellular Proliferation:

In certain preferred embodiments of the present disclosure, a method for screening test compounds for their effect on cellular proliferation is provided. This method may include measuring cellular proliferation changes in a patient during the course of treatment for a disease with a specific compound.

1. “Double Click Reaction”:

According to certain embodiments of the present disclosure, methods for identifying an agent that perturbs cellular proliferation are provided, the methods comprising:
a) contacting a cell with a test agent;
b) contacting the cell with an effective amount of an alkynyl-modified nucleoside analogue, such that the alkynyl-modified nucleoside analogue is incorporated into DNA of the cell;
c) contacting the cell with an adapter linker of structural formula (I) under conditions such that the alkynyl moiety of the alkynyl-modified nucleoside analogue forms a covalent link with the azide moiety of the adapter linker, thereby forming an adapter intermediate;
d) contacting the cell with a detectable label comprising a cycloalkene group under conditions such that a covalent link forms between the tetrazine functional group of the adapter intermediate and the cycloalkene group of the detectable label;
e) measuring the amount of detectable label incorporated into the DNA, wherein the amount of label indicates the extent of cellular proliferation; and
f) identifying the test agent as an agent that perturbs cellular proliferation if the amount of label measured in step (e) is less than or greater than the amount of label measured in a control application in which the cell is not contacted with the test agent.

In certain embodiments, the present disclosure provides for methods of identifying an agent that perturbs cellular proliferation in an organism, the methods comprising:
a) exposing an organism to a test agent;
b) administering to the organism an effective amount of an alkynyl-modified nucleoside analogue, such that the alkynyl-modified nucleoside analogue is incorporated into DNA of cells of the organism;
c) contacting at least one cell of the organism with an adapter linker of structural formula (I) under conditions such that the alkynyl moiety of the alkynyl-modified nucleoside analogue forms a covalent link with the azide moiety of the adapter linker, thereby forming an adapter intermediate;
d) contacting the at least one cell of the organism with a detectable label comprising a cycloalkene group under conditions such that a covalent link forms between the tetrazine functional group of the adapter intermediate and the cycloalkene group of the detectable label;
e) measuring the amount of detectable label incorporated into the DNA, wherein the amount of label indicates the extent of cellular proliferation; and
f) identifying the test agent as an agent that perturbs cellular proliferation in the organism if the amount of label measured in step (e) is less than or greater than the amount of label measured in a control application in which the organism is not exposed to the test agent.

In certain embodiments, the alkynyl-modified nucleoside analogue is an EdU or an EdC. In certain embodiments, the cycloalkene group is a trans-cyclooctene or a cyclopentene. In certain embodiments, the detectable label is a colorimetric label. In certain embodiments, the colorimetric label is selected from horseradish peroxidase, alkaline phosphatase, beta-galactosidase, glucose oxidase or beta-lactamase. In certain embodiments, the horseradish peroxidase is a trans-cyclooctene-horseradish peroxidase conjugate. In certain embodiments, the cell is in a multi-well plate.

In certain embodiments, the step of contacting the cell with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the adapter linker is performed in the presence of a copper chelator.

2. “Fluorescent Intermediate Reaction”:

According to certain embodiments of the present disclosure, methods for identifying an agent that perturbs cellular proliferation are provided, the methods comprising:
a) contacting a cell with a test agent;
b) contacting the cell with an effective amount of an alkynyl-modified nucleoside analogue, such that the alkynyl-modified nucleoside analogue is incorporated into DNA of the cell;
c) contacting the cell with an azide-modified fluorescent dye under conditions such that the alkynyl moiety of the alkynyl-modified nucleoside analogue forms a covalent link with the azide moiety of the fluorescent dye;
d) contacting the cell with an anti-fluorescent dye antibody that binds to the azide-modified fluorescent dye;
e) contacting the cell with a secondary antibody conjugated to a detectable label, wherein the secondary antibody binds to the anti-fluorescent dye antibody;
f) measuring the amount of detectable label, wherein the amount of label indicates the extent of cellular proliferation; and
g) identifying the test agent as an agent that perturbs cellular proliferation if the amount of label measured in step (f) is less than or greater than the amount of label measured in a control application in which the cell is not contacted with the test agent.

According to certain embodiments of the present disclosure, methods for identifying an agent that perturbs cellular proliferation are provided, the methods comprising:
a) contacting a cell with a test agent;
b) contacting the cell with an effective amount of an alkynyl-modified nucleoside analogue, such that the alkynyl-modified nucleoside analogue is incorporated into DNA of the cell;
c) contacting the cell with an azide-modified fluorescent dye under conditions such that the alkynyl moiety of the alkynyl-modified nucleoside analogue forms a covalent link with the azide moiety of the fluorescent dye;
d) contacting the cell with an anti-fluorescent dye antibody conjugated to a detectable label, wherein the antibody binds to the azide-modified fluorescent dye;
e) measuring the amount of detectable label, wherein the amount of label indicates the extent of cellular proliferation; and
f) identifying the test agent as an agent that perturbs cellular proliferation if the amount of label measured in step (e) is less than or greater than the amount of label measured in a control application in which the cell is not contacted with the test agent.

In certain embodiments of the present disclosure, methods for identifying an agent that perturbs cellular proliferation in an organism are provided, the methods comprising:
a) exposing an organism to a test agent;
b) administering to the organism an effective amount of an alkynyl-modified nucleoside analogue, such that the alkynyl-modified nucleoside analogue is incorporated into DNA of cells of the organism;
c) contacting at least one cell of the organism with an azide-modified fluorescent dye under conditions such that the alkynyl moiety of the alkynyl-modified nucleoside analogue forms a covalent link with the azide moiety of the fluorescent dye;
d) contacting the at least one cell of the organism with an anti-fluorescent dye antibody that binds to the azide-modified fluorescent dye;
e) contacting the at least one cell of the organism with a secondary antibody conjugated to a detectable label, wherein the secondary antibody binds to the anti-fluorescent dye antibody;
f) measuring the amount of detectable label, wherein the amount of label indicates the extent of cellular proliferation; and
g) identifying the test agent as an agent that perturbs cellular proliferation if the amount of label measured in step (f) is less than or greater than the amount of label measured in a control application in which the organism is not exposed to the test agent.

According to certain embodiments of the present disclosure, methods for identifying an agent that perturbs cellular proliferation in an organism are provided, the methods comprising:
a) exposing an organism to a test agent;
b) administering to the organism an effective amount of an alkynyl-modified nucleoside analogue, such that the alkynyl-modified nucleoside analogue is incorporated into DNA of cells of the organism;
c) contacting at least one cell of the organism with an azide-modified fluorescent dye under conditions such that the alkynyl moiety of the alkynyl-modified nucleoside analogue forms a covalent link with the azide moiety of the fluorescent dye;
d) contacting the at least one cell of the organism with an anti-fluorescent dye antibody conjugated to a detectable label, wherein the antibody binds to the azide-modified fluorescent dye;
e) measuring the amount of detectable label, wherein the amount of label indicates the extent of cellular proliferation; and
f) identifying the test agent as an agent that perturbs cellular proliferation if the amount of label measured in step (e) is less than or greater than the amount of label measured in a control application in which the organism is not exposed to the test agent.

In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the cell with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified fluorescent dye is performed in the presence of a copper chelator.

3. “Biotin Intermediate Method”:

According to certain embodiments of the present disclosure, methods for identifying an agent that perturbs cellular proliferation are provided, the methods comprising:
a) contacting a cell with a test agent;
b) contacting the cell with an effective amount of an alkynyl-modified nucleoside analogue, such that the alkynyl-modified nucleoside analogue is incorporated into DNA of the cell;
c) contacting the cell with an azide-modified biotin under conditions such that the alkynyl moiety of the alkynyl-modified nucleoside analogue forms a covalent link with the azide moiety of the biotin;
d) contacting the cell with a (strept)avidin conjugated to a detectable label;
e) measuring the amount of detectable label, wherein the amount of label indicates the extent of cellular proliferation; and
f) identifying the test agent as an agent that perturbs cellular proliferation if the amount of label measured in step (e) is less than or greater than the amount of label measured in a control application in which the cell is not contacted with the test agent.

According to certain embodiments of the present disclosure, methods for identifying an agent that perturbs cellular proliferation in an organism are provided, the methods comprising:
a) exposing an organism to a test agent;
b) administering to the organism an effective amount of an alkynyl-modified nucleoside analogue, such that the alkynyl-modified nucleoside analogue is incorporated into DNA of cells of the organism;
c) contacting at least one cell of the organism with an azide-modified biotin under conditions such that the alkynyl moiety of the alkynyl-modified nucleoside analogue forms a covalent link with the azide moiety of the biotin;
d) contacting the at least one cell of the organism with a (strept)avidin conjugated to a detectable label;
e) measuring the amount of detectable label, wherein the amount of label indicates the extent of cellular proliferation; and
f) identifying the test agent as an agent that perturbs cellular proliferation if the amount of label measured in step (e) is less than or greater than the amount of label measured in a control application in which the organism is not exposed to the test agent.

In certain embodiments, the alkynyl-modified nucleoside analogue is an EdU or an EdC. In certain embodiments, the detectable label is a colorimetric label. In certain embodiments, the detectable label is selected from horseradish peroxidase, alkaline phosphatase, beta-galactosidase, glucose oxidase and beta-lactamase.

In certain embodiments, the step of contacting the cell with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified biotin is performed in the presence of a copper chelator.

A large number of diseases and disorders are known to be characterized by altered cellular proliferation rates and thus can be monitored by methods of the present disclosure. Such diseases and disorders include, but are not limited to, malignant tumors of any type (e.g., breast, lung, colon, skin, lymphoma, leukemia, and the like); precancerous conditions (e.g., adenomas, polyps, prostatic hypertrophy, ulcerative colitis, and the like); immune disorders such as AIDS, autoimmune disorders, and primary immunodeficiencies; hematologic conditions such as white blood cell deficiencies (e.g., granulocytopenia), anemias of any type, myeloproliferative disorders, lymphoproliferative disorders and the like; organ failure such as alcoholic and viral hepatitis, diabetic nephropathy, myotrophic conditions, premature gonadal failure and the like; conditions affecting bones and muscles, such as osteoporosis; endocrine conditions such as diabetes, hypothyroidism and hyperthyroidism, polycystic ovaries and the like; infectious diseases, such as tuberculosis, bacterial infections, abscesses and other localized tissue infections, viral infections and the like; and vascular disorders, such as atherogenesis, cardiomyopathies, and the like.

Cancer cells can be removed from a patient and grown in culture. A baseline DNA synthesis rate can be determined with a first nucleoside analog pulse, then a drug is added along with a second nucleoside analog and the change of DNA synthesis rate determined, which in the case of drug resistance/sensitivity in cancer cells would easily be determined. Screens for compounds which either stimulate or block DNA synthesis at various places in the cell cycle could be greatly improved by the addition having an accurate baseline synthesis measurement which does not alter the state of the cell proliferation.

For example, breast cancer cells are removed from a patient and grown in culture. The baseline cellular proliferation rate may be established by adding a first pulse label of EdU. Then, the cells may be treated with a chemotherapy drug, for example tamoxifen, and treated with a second pulse label of BrdU. The cellular proliferation rate in response to tamoxifen is then measured by comparing incorporation of EdU to BrdU. This process may be repeated over the course of the breast cancer patient's treatment to ensure that the patient's cancer cells remain responsive to the chosen chemotherapeutic agent, in this case, tamoxifen. In this present example, the clinician would be looking for a decrease in cellular proliferation upon treatment with the chemotherapy drug. Once the dual pulse labeling of DNA in the breast cancer patient's cells demonstrated no change in cellular proliferation upon treatment with a particular drug, the clinician could reevaluate whether the patient would benefit from continued treatment with that drug or should be switched to a different chemotherapeutic agent.

In still further embodiments, the present disclosure provides a method for identifying new compounds, which may be termed as “test compounds”, which have a desired effect on cellular proliferation. Depending on the application, this desired effect may be to stimulate, to inhibit, or to not affect cellular proliferation.

In another aspect, the present disclosure provides methods for the identification of agents that perturb cellular proliferation. These methods may be used for screening agents for their ability to induce (i.e., increase, enhance or otherwise exacerbate) or inhibit (i.e., decrease, slow down or otherwise suppress) cell proliferation.

The manner of performing the steps of contacting the cell; the staining reagent; the label type; and methods of detecting the labeled nucleic acid polymers are analogous to those described for other methods of the present disclosure relating to measuring cellular proliferation and cellular proliferation rates in cells in vitro. As will be appreciated by one of ordinary skill in the art, the screening methods of the present disclosure may also be used to identify compounds or agents that regulate cellular proliferation (i.e., compounds or agents that can decrease, slow down or suppress proliferation of over-proliferative cells or that can increase, enhance or exacerbate proliferation of under-proliferative cells).

The screening assays of the present disclosure may be performed using any normal or transformed cells that can be grown in standard tissue culture plastic ware. Cells may be primary cells, secondary cells, or immortalized cells. Preferably, cells to be used in the inventive screening methods are of mammalian (e.g., human or animal) origin. Cells may be from any organ or tissue origin and of any cell types, as described above.

Selection of a particular cell type and/or cell line to perform a screening assay according to the present disclosure will be governed by several factors such as the nature of the agent to be tested and the intended purpose of the assay. For example, a toxicity assay developed for primary drug screening (i.e., first round(s) of screening) may preferably be performed using established cell lines, which are commercially available and usually relatively easy to grow, while a toxicity assay to be used later in the drug development process may preferably be performed using primary or secondary cells, which are often more difficult to obtain, maintain, and/or grow than immortalized cells but which represent better experimental models for in vivo situations.

In certain embodiments, the screening methods are performed using cells contained in a plurality of wells of a multi-well assay plate. Such assay plates are commercially available, for example, from Strategene Corp. (La Jolla, Calif.) and Corning Inc. (Acton, Mass.), and include, for example, 48-well, 96-well, 384-well and 1536-well plates.

As will be appreciated by those of ordinary skill in the art, any kind of compounds or agents can be tested using the methods provided herein. A test compound may be a synthetic or natural compound; it may be a single molecule, a mixture of different molecules or a complex of different molecules. In certain embodiments, the methods provided herein are used for testing one or more compounds. In other embodiments, the methods provided herein are used for screening collections or libraries of compounds.

Compounds that can be tested for their capacity or ability to perturb (i.e., induce or inhibit) or regulate cell proliferation can belong to any of a variety of classes of molecules including, but not limited to, small molecules, peptides, saccharides, steroids, antibodies (including fragments or variants thereof), fusion proteins, antisense polynucleotides, ribozymes, small interfering RNAs, peptidomimetics, and the like.

Compounds or agents to be tested according to methods of the present disclosure may be known or suspected to perturb or regulate cell proliferation. Alternatively, the assays may be performed using compounds or agents whose effects on cell proliferation are unknown.

Examples of compounds that may affect cell proliferation and that can be tested by the methods of the present disclosure include, but are not limited to, carcinogens; toxic agents; chemical compounds such as solvents; mutagenic agents; pharmaceuticals; particulates, gases and noxious compounds in smoke (including smoke from cigarette, cigar and industrial processes); food additives; biochemical materials; hormones; pesticides; ground-water toxins; and environmental pollutants. Examples of agents that may affect cell proliferation and that can be tested by the methods of the present disclosure include, but are not limited to, microwave radiation, electromagnetic radiation, radioactive radiation, ionizing radiation, heat, and other hazardous conditions produced by or present in industrial or occupational environments.

According to screening methods of the present disclosure, determination of the ability of a test agent to perturb or regulate cellular proliferation includes comparison of the amount of label incorporated into DNA of a cell that has been contacted with the test agent with the amount of label incorporated into DNA of a cell that has not been contacted with the test agent.

A test agent is identified as an agent that perturbs cellular proliferation if the amount of label incorporated into DNA of the cell that has been contacted with the test agent is less than or greater than the amount of label measured in the control cell. More specifically, if the amount of label incorporated into DNA of the cell that has been contacted with the test agent is less than the amount of label measured in the control cell, the test agent is identified as an agent that inhibits cell proliferation. If the amount of label incorporated into DNA of the cell that has been contacted with the test agent is greater than the amount of label measured in the control cell, the test agent is identified as an agent that induces cell proliferation.

Reproducibility of the results may be tested by performing the analysis more than once with the same concentration of the test agent (for example, by incubating cells in more than one well of an assay plate). Additionally, since a test agent may be effective at varying concentrations depending on the nature of the agent and the nature of it mechanism(s) of action, varying concentrations of the test agent may be tested (for example, added to different wells containing cells). Generally, test agent concentrations from 1 fM to about 10 mM are used for screening. Preferred screening concentrations are between about 10 pM and about 100 μM.

In certain embodiments, the methods provided herein further involve the use of one or more negative or positive control compounds. A positive control compound may be any molecule or agent that is known to perturb (i.e., induce or inhibit) or regulate cellular proliferation. A negative control compound may be any molecule or agent that is known to have no detectable effects on cellular proliferation. In these embodiments, the methods provided herein further comprise comparing the effects of the test agent to the effects (or absence thereof) of the positive or negative control compound.

As will be appreciated by those skilled in the art, it is generally desirable to further characterize an agent identified by the screening methods provided herein as an agent that perturbs or an agent that regulates cellular proliferation. For example, if a test compound has been identified as an agent that perturbs (or regulates) cellular proliferation using a given cell culture system (e.g., an established cell line), it may be desirable to test this ability in a different cell culture system (e.g., primary or secondary cells).

Test agents identified by the screening methods of the present disclosure may also be further tested in assays that allow for the determination of the agents' properties in vivo.

As will be appreciated by one of ordinary skill in the art, these methods can be used to identify agents that regulate cellular proliferation in vivo.

The manner of administration, staining reagent, type of label and method of detection of the labeled nucleic acid polymers are analogous to those described herein for other methods of the present disclosure relating to measuring cellular proliferation in living systems.

TUNEL Assays for Measuring Apoptosis:

Since the introduction of terminal deoxynucleotidyl transferase-dUTP nick end labeling (TUNEL) assay (Gavrieli et al., J. Cell. Biol. 119:493 (1992)), the TUNEL assay is the most widely used in situ test for apoptosis study (Huerta et al., J. Surg. Res. 139:143 (2007)). The TUNEL assay is based on the incorporation of modified dUTPs by the enzyme terminal deoxynucleotidyl transferase (TdT) at the 3′-OH ends of fragmented DNA, a hallmark as well as the ultimate determinate of apoptosis. The modifications are typically fluorophores or haptens, including biotin or bromine which can be detected directly in the case of a fluorescently-modified nucleotide (i.e., fluorescein-dUTP), or indirectly with streptavidin or antibodies, if biotin-dUTP or BrdUTP are used, respectively. Often at late stages of apoptosis, adherent cells are known to detach or “pop” off. For a reliable and reproducible TUNEL imaging assay, the modified nucleotide must not only be an acceptable substrate for TdT, but the detection method must also be sensitive without bringing about any additional loss of cells from the sample.

In certain embodiments of the present disclosure, the TUNEL assays use an alkynyl-modified nucleotide, such as EdUTP, which is incorporated at the 3′-OH ends of fragmented DNA by the TdT enzyme. After the incorporation of the alkynyl-modified nucleotide at the site of DNA fragmentation, detection of apoptosis can be performed using any of the detection methods provided herein: the “Double Click Reaction”, the “Fluorescent Intermediate Method” or the “Biotin Intermediate Method” described hereinabove. Because of the high degree of labeling specificity inherent in the click reaction and the small size of the alkynyl moiety, the alkynyl-modified nucleotide is readily incorporated by TdT.

In certain embodiments of the present disclosure, methods for detecting apoptosis are provided which utilize a dNTP modified with an alkyne, such as EdUTP, EdCTP, EdATP and EdTTP.

1. “Double Click Reaction”:

According to certain embodiments of the present disclosure, methods for detecting apoptosis are provided, the methods comprising:
a) contacting a cell with an effective amount of an alkynyl-modified nucleoside analogue and a terminal deoxynucleotidyl transferase (TdT), such that the alkynyl-modified nucleoside analogue is incorporated into DNA of the cell;
b) contacting the cell with an adapter linker of structural formula (I) under conditions such that the alkynyl moiety of the alkynyl-modified nucleoside analogue forms a covalent link with the azide moiety of the adapter linker, thereby forming an adapter intermediate;
c) contacting the cell with a detectable label comprising a cycloalkene group under conditions such that a covalent link forms between the tetrazine functional group of the adapter intermediate and the cycloalkene group of the detectable label; and
d) measuring the amount of detectable label incorporated into the DNA, wherein the amount of label indicates the presence of apoptosis.

In certain embodiments, the alkynyl-modified nucleoside analogue is an EdUTP or an EdCTP. In certain embodiments, the cycloalkene group is a trans-cycloalkene or a cyclopropene. In certain embodiments, the detectable label is a colorimetric label. In certain embodiments, the colorimetric label is selected from horseradish peroxidase, alkaline phosphatase, beta-galactosidase, glucose oxidase or beta-lactamase. In certain embodiments, the horseradish peroxidase is a trans-cyclooctene-horseradish peroxidase conjugate. In certain embodiments, the cell is in a multi-well plate.

In certain embodiments, the step of contacting the cell with the adapter linker is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the adapter linker is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the adapter linker is performed in the presence of a copper chelator.

2. “Fluorescent Intermediate Method”:

According to certain embodiments of the present disclosure, methods for detecting apoptosis are provided, the methods comprising:
a) contacting a cell with an effective amount of an alkynyl-modified nucleoside analogue and a terminal deoxynucleotidyl transferase (TdT), such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody that binds to the azide-modified fluorescent dye;
d) contacting the cell with a secondary antibody conjugated to a detectable label, wherein the secondary antibody binds to the anti-fluorescent dye antibody; and
e) measuring the amount of detectable label, wherein the amount of label indicates the presence of apoptosis.

In certain embodiments provided herein, methods for detecting apoptosis are provided, the methods comprising:
a) contacting a cell with an effective amount of an alkynyl-modified nucleoside analogue and a terminal deoxynucleotidyl transferase (TdT), such that the alkynyl-modified nucleoside analogue is incorporated into DNA of the cell;
b) contacting the cell with an azide-modified fluorescent dye under conditions such that the alkynyl moiety of the alkynyl-modified nucleoside analogue forms a covalent link with the azide moiety of the fluorescent dye;
c) contacting the cell with an anti-fluorescent dye antibody conjugated to a detectable label, wherein the antibody binds to the azide-modified fluorescent dye; and
d) measuring the amount of detectable label, wherein the amount of label indicates the presence of apoptosis.

In certain embodiments, the alkynyl-modified nucleoside analogue is an EdUTP or an EdCTP. In certain embodiments, the detectable label is a colorimetric label. In certain embodiments, the detectable label is selected from horseradish peroxidase, alkaline phosphatase, beta-galactosidase, glucose oxidase or beta-lactamase. In certain embodiments, the azide-modified fluorescent dye is selected from a xanthene dye, a cyanine dye, a coumarin dye and a pyrene dye.

In certain embodiments, the step of contacting the cell with the azide-modified fluorescent dye is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified fluorescent dye is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified fluorescent dye is performed in the presence of a copper chelator.

3. “Biotin Intermediate Method”:

According to certain embodiments of the present disclosure, methods of detecting apoptosis are provided, the methods comprising:
a) contacting a cell with an effective amount of an alkynyl-modified nucleoside analogue and a terminal deoxynucleotidyl transferase (TdT), such that the alkynyl-modified nucleoside analogue is incorporated into DNA of the cell;
b) contacting the cell with an azide-modified biotin under conditions such that the alkynyl moiety of the alkynyl-modified nucleoside analogue forms a covalent link with the azide moiety of the biotin;
c) contacting the cell with a (strept)avidin conjugated to a detectable label; and
d) measuring the amount of detectable label, wherein the amount of label indicates the presence of apoptosis.

In certain embodiments, the alkynyl-modified nucleoside analogue is an EdUTP or an EdCTP. In certain embodiments, the detectable label is a colorimetric label. In certain embodiments, the colorimetric label is selected from horseradish peroxidase, alkaline phosphatase, beta-galactosidase, glucose oxidase and beta-lactamase.

In certain embodiments, the step of contacting the cell with the azide-modified biotin is performed in the presence of copper in the Cu(I) reduction state. In certain embodiments, the Cu(I) is a cuprous salt. In certain embodiments, the cuprous salt is a cuprous halide. In certain embodiments, the step of contacting the cell with the azide-modified biotin is performed in the presence of copper in the Cu(II) reduction state and a reducing agent. In certain embodiments, the Cu(II) is Cu(NO₃)₂, Cu(OAc)₂ or CuSO₄. In certain embodiments, the step of contacting the cell with the azide-modified biotin is performed in the presence of a copper chelator.

Labeling of RNA and RNA Localization Studies:

As described hereinabove, the labeling methods of the present disclosure may be used for labeling RNA. In such methods, the ribonucleotide polymer comprising the nucleotide analogue may be prepared by any suitable method, as known in the art. For example, the ribonucleotide polymer may be synthesized by in vitro transcription of DNA, cloned downstream of T3, T7 or SP6 polymerases promoters in the presence of nucleotide triphosphates (including the nucleotide analogue triphosphate) as substrates. Alternatively, the ribonucleotide polymer may be prepared using amplification methods.

The labeling methods provided herein may be used in microarray hybridization assays to measure mRNA transcript levels of many genes in parallel. The labeling methods provided herein may also find applications in ribosome display, a cell-free system for the in vitro selection of proteins and peptides (Tuerk and Gold, Science, 249:505-510 (1990); Joyce, Gene, 82:83-87 (1989); Szostak, Trends Biochem. Sci., 17:89-93 (1992); Tsai et al., Proc. Natl. Acad. Sci. USA, 89:8864-8868 (1992); Doudna et al., Proc. Natl. Acad. Sci. USA, 92:2355-2359 (1995); Shaffitzel et al., J. Immunol. Methods, 231:119-135 (1999); Lipovsel and Pluckthun, J. Immunol. Methods, 290:51-67 (2001); Jackson et al., Brief Funct. Genomic Proteomic, 2:308-319 (2004)). These selection assays generally involve adding an RNA library to the protein or molecule of interest, washing to remove unbound RNA, and specifically eluting the RNA bound to the protein. The RNA is then reversed transcribed and amplified by PCR. The cDNA obtained is then transcribed in the presence of nucleotide analogues for detection purposes. Those molecules that are found to bind the protein or other molecule of interest are cloned and sequenced to look for common sequences. The common sequence is then used to develop therapeutic oligonucleotides.

The RNA labeling methods of the present disclosure may also be used for visualizing mRNA movement (transport and localization) in living cells. mRNA localization is a common mode of post-transcriptional regulation of gene expression that targets a protein to its site of function (Palacios and St Johnston, Annu. Rev. Cell Dev. Biol., 17:569-614 (2001); Jansen, Nature Rev. Mol. Cell Biol., 2:247-256 (2001); Kloc et al., Cell, 108:533-544 (2002)). Many of the best characterized localized mRNAs are found in oocytes and early embryos, where they function as localized determinants that control axis formation and the development of the germline. mRNA localization has also been shown to play an important role in somatic cells, such as neurons, where it may be involved in learning and memory. Different mRNA visualization methods have been developed to identify the machinery and mechanisms involved in mRNA transport and localization, including aminoallyl-uridine triphosphate incorporation into RNA followed by fluorescein or rhodamine coupling and direct incorporation of ALEXA FLUOR™-uridine triphosphate into RNA. (Van de Bor and Davis, Curr. Opin. Cell Biol, 16:300-307 (2004)). mRNA molecules fluorescently labeled in vitro according to the present disclosure may be introduced into living cells and their movement monitored in real time.

Labels:

As already mentioned above, the role of a label or reporter molecule is to allow visualization or detection of a nucleic acid polymer, e.g., DNA in a cell, following labeling. Preferably, a label (or detectable agent or moiety) is selected such that it generates a signal which can be measured and whose intensity is related (e.g., proportional) to the amount of labeled nucleic acid polymer, e.g., in a sample being analyzed.

A label used in a labeling reagent in the methods and compositions described herein, is any chemical moiety, organic or inorganic, that is, for example, colorimetric, and retains its enzymatic and/or colorimetric properties when covalently attached to a modified nucleoside such as, by way of example only, an azide, and alkyne or a phosphine.

The selection of a particular label will depend on the purpose of the labeling to be performed and will be governed by several factors, such as the ease and cost of the labeling method, the quality of sample labeling desired, the effects of the detectable moiety on the cell or organism, the nature of the detection system, the nature and intensity of the signal generated by the detectable moiety, and the like.

The labels or reporter molecules used in the methods and compositions provided herein include any directly or indirectly detectable reporter molecule known by one skilled in the art that can be covalently attached to a modified nucleic acid described herein. In certain embodiments, the labels used in the methods and compositions provided herein include any directly or indirectly detectable label known by one skilled in the art that can be covalently attached to an azide modified nucleic acid, an alkyne modified nucleic acid or a phosphine modified nucleic acid.

Labels used in the methods and compositions described herein can contain, but are not limited to, a chromophore, a fluorophore, a fluorescent protein, a phosphorescent dye, a tandem dye, a particle, a hapten, an enzyme and a radioisotope. In certain embodiments, such labels include colorimetric compounds, tags, chromophores, haptens, and enzymes.

Enzymes find use as labels for the detection reagents/reporter molecules used in the methods and compositions described herein. Enzymes are desirable labels because amplification of the detectable signal can be obtained resulting in increased assay sensitivity. The enzyme itself does not produce a detectable response but functions to break down a substrate when it is contacted by an appropriate substrate such that the converted substrate produces a fluorescent, colorimetric or luminescent signal. Enzymes amplify the detectable signal because one enzyme on a labeling reagent can result in multiple substrates being converted to a detectable signal. This is advantageous where there is a low quantity of target present in the sample or a fluorophore does not exist that will give comparable or stronger signal than the enzyme. The enzyme substrate is selected to yield the preferred measurable product, e.g. colorimetric, fluorescent or chemiluminescence. Such substrates are extensively used in the art, many of which are described in Molecular Probes Handbook of Fluorescent Probes and Research Chemicals, supra.

In certain embodiments, colorimetric substrate and enzyme combinations use oxidoreductases such as, by way of example only, horseradish peroxidase (HRP) and a substrate such as, by way of example only, 3,3′-diaminobenzidine (DAB) or 3-amino-9-ethylcarbazole (AEC), which yield a distinguishing color (brown and red, respectively). Other colorimetric oxidoreductase substrates used with the enzymatic reporter molecules described herein include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates used with the enzymatic reporter molecules described herein include, but are not limited to, homovanillic acid or 4-hydroxy-3-methoxyphenylacetic acid, reduced phenoxazines and reduced benzothiazines, including AMPLEX™ Red reagent and its variants (U.S. Pat. No. 4,384,042), AMPLEX™ UltraRed and its variants (PCT Publication No. WO 05/42504) and reduced dihydroxanthenes, including dihydrofluoresceins (U.S. Pat. No. 6,162,931) and dihydrorhodamines including dihydrorhodamine 123. Peroxidase substrates can be used with the enzymatic reporter molecules described herein. Such peroxide substrates include, but are not limited to, tyramides (U.S. Pat. Nos. 5,196,306; 5,583,001 and 5,731,158) which represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

In other embodiments, the colorimetric (and in some cases fluorogenic) substrates and enzymes combination used in reporter molecules described herein include a phosphatase enzyme such as, by way of example only, an acid phosphatase, an alkaline phosphatase or a recombinant version of such a phosphatase. A colorimetric substrate used in combination with such phosphatases includes, but are not limited to, 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912), fluorescein diphosphate, 3-o-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates (U.S. Pat. Nos. 5,316,906 and 5,443,986).

Other enzymes used in labels described herein include glycosidases, including, but not limited to, beta-galactosidase, beta-glucuronidase and beta-glucosidase. The colorimetric substrates used with such enzymes include, but are not limited to, 5-bromo-4-chloro-3-indolyl beta-D-galactopyranoside (X-gal) and similar indolyl galactosides, glucosides, and glucuronides, o-nitrophenyl beta-D-galactopyranoside (ONPG) and p-nitrophenyl beta D-galactopyranoside. Preferred fluorogenic substrates include resorufin beta-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236), 4-methylumbelliferyl beta D-galactopyranoside, carboxyumbelliferyl beta-D-galactopyranoside and fluorinated coumarin beta-D-galactopyranosides (U.S. Pat. No. 5,830,912).

Additional enzymes used in labels described herein include, but are not limited to, hydrolases such as cholinesterases and peptidases, oxidases such as glucose oxidase and cytochrome oxidases, reductases for which suitable substrates are known, and beta-lactamase.

Enzymes and their appropriate substrates that produce chemiluminescence can also be used in labels or reporter molecules used in the methods described herein. Such enzymes include, but are not limited to, natural and recombinant forms of luciferases and aequorins. In addition, the chemiluminescence-producing substrates for phosphatases, glycosidases and oxidases such as those containing stable dioxetanes, luminol, isoluminol and acridinium esters an also be used in reporter molecules described herein.

In addition to enzymes, haptens can be used in label/reporter molecules described herein. In certain embodiments, such haptens include hormones, naturally occurring and synthetic drugs, pollutants, allergens, affector molecules, growth factors, chemokines, cytokines, lymphokines, amino acids, peptides, chemical intermediates, nucleotides, digoxin, biotin and the like. Biotin is useful because it can function in an enzyme system to further amplify the detectable signal, and it can function as a tag to be used in affinity chromatography for isolation purposes. For detection purposes, an enzyme conjugate that has affinity for biotin is used, such as, by way of example only, avidin-horseradish peroxidase or streptavidin-horseradish peroxidase. Subsequently a peroxidase substrate as described herein can be added to produce a detectable signal.

A fluorophore used in labels in the methods and compositions described herein, can contain one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

In addition to colorimetric detection, fluorophores also find use as labels or reporter molecules in the methods and compositions described herein, for example, as intermediates in a “Fluorescence Intermediate Method” provided herein. In certain embodiments, the fluorophore is any chemical moiety that exhibits an absorption maximum at wavelengths greater than 280 nm, and retains its spectral properties when covalently attached to a modified nucleotide such as, by way of example only, an azide, and alkyne or a phosphine. Fluorophores used in labels in the methods and compositions described herein include, without limitation; a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Patent Application Publication Nos. 2002-0077487 and 2002-0064794), a carbocyanine (including any corresponding compounds in U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; 6,664,047; 6,974,873; 6,977,305; PCT Publication Nos. WO 02/26891, WO 97/40104, WO 99/51702, WO 01/21624; and European Patent Application Publication No. EP 1 065 250 A1), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 6,716,979 and 5,451,343), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

Many fluorophores can also function as chromophores and thus the described fluorophores are also chromophores used in labels in the methods and compositions described herein.

Fluorescent proteins can also be used in label/reporter molecules described herein for use in the methods, compositions and modified nucleic acids described herein. Non-limiting examples of such fluorescent proteins include green fluorescent protein (GFP) and the phycobiliproteins and the derivatives thereof. The fluorescent proteins, especially phycobiliprotein, are particularly useful for creating tandem dye labeled modified nucleic acids. These tandem dyes comprise a fluorescent protein and a fluorophore for the purposes of obtaining a larger stokes shift wherein the emission spectra is farther shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. The fluorescent protein and fluorophore function as an energy transfer pair wherein the fluorescent protein emits at the wavelength that the fluorophore absorbs and the fluorophore then emits at a wavelength farther from the fluorescent proteins emission wavelength than could have been obtained with only the fluorescent protein. A particularly useful combination is the phycobiliproteins disclosed in U.S. Pat. Nos. 4,520,110; 4,859,582; 5,055,556 and the sulforhodamine fluorophores disclosed in U.S. Pat. No. 5,798,276, or the sulfonated cyanine fluorophores disclosed in U.S. Pat. Nos. 6,977,305 and 6,974,873; or the sulfonated xanthene derivatives disclosed in U.S. Pat. No. 6,130,101 and those combinations disclosed in U.S. Pat. No. 4,542,104. Alternatively, the fluorophore functions as the energy donor and the fluorescent protein is the energy acceptor.

Carrier Molecules:

In the methods and compositions described herein the modified nucleic acids can be conjugated to a carrier molecule. In certain embodiments, the modified nucleic acids contain at least one alkyne moiety capable of reacting with a carrier molecule containing an azide moiety.

A variety of carrier molecules can be used in the methods and compositions described herein, including, but not limited to, antigens, steroids, vitamins, drugs, haptens, metabolites, toxins, environmental pollutants, amino acids, peptides, proteins, nucleic acids, nucleic acid polymers, carbohydrates, lipids, and polymers. In certain embodiments, the carrier molecule contains an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus or combinations thereof.

In other embodiments, the carrier molecule is selected from a hapten, a nucleotide, an oligonucleotide, a nucleic acid polymer, a protein, a peptide or a polysaccharide. In still other embodiments, the carrier molecule is an amino acid, a peptide, a protein, a polysaccharide, a nucleoside, a nucleotide, an oligonucleotide, a nucleic acid, a hapten, a psoralen, a drug, a hormone, a lipid, a lipid assembly, a tyramine, a synthetic polymer, a polymeric microparticle, a biological cell, cellular components, an ion chelating moiety, an enzymatic substrate or a virus. In further embodiments, the carrier molecule is an antibody or fragment thereof, an antigen, an avidin or streptavidin, a biotin, a dextran, an IgG binding protein, a fluorescent protein, agarose, and a non-biological microparticle.

In certain embodiments wherein the carrier molecule is an enzymatic substrate, the enzymatic substrate is selected from an amino acid, a peptide, a sugar, an alcohol, alkanoic acid, 4-guanidinobenzoic acid, a nucleic acid, a lipid, sulfate, phosphate, —CH₂OCO-alkyl and combinations thereof. In certain embodiments, such enzyme substrates can be cleaved by enzymes selected from peptidases, phosphatases, glycosidases, dealkylases, esterases, guanidinobenzotases, sulfatases, lipases, peroxidases, histone deacetylases, exonucleases, reductases, endoglycoceramidases and endonucleases.

In other embodiments, the carrier molecule is an amino acid (including those that are protected or are substituted by phosphates, carbohydrates, or C₁ to C₂₂ carboxylic acids), or a polymer of amino acids such as a peptide or protein. In a related embodiment, the carrier molecule contains at least five amino acids, more preferably 5 to 36 amino acids. Such peptides include, but are not limited to, neuropeptides, cytokines, toxins, protease substrates, and protein kinase substrates. Other peptides may function as organelle localization peptides, that is, peptides that serve to target the conjugated compound for localization within a particular cellular substructure by cellular transport mechanisms, including, but not limited to, nuclear localization signal sequences. In certain embodiments, the protein carrier molecules include enzymes, antibodies, lectins, glycoproteins, histones, albumins, lipoproteins, avidin, streptavidin, protein A, protein G, phycobiliproteins and other fluorescent proteins, hormones, toxins and growth factors. In other embodiments, the protein carrier molecule is an antibody, an antibody fragment, avidin, streptavidin, a toxin, a lectin, or a growth factor. In further embodiments, the carrier molecules contain haptens including, but not limited to, biotin, digoxin, digoxigenin and fluorophores.

The carrier molecules used in the methods and composition described herein can also contain a nucleic acid base, nucleoside, nucleotide or a nucleic acid polymer, optionally containing an additional linker or spacer for attachment of a fluorophore or other ligand, such as an alkynyl linkage (U.S. Pat. No. 5,047,519), an aminoallyllinkage (U.S. Pat. No. 4,711,955) or other linkage. In other embodiments, the nucleotide carrier molecule is a nucleoside or a deoxynucleoside or a dideoxynucleoside, while in other embodiments, the carrier molecule contains a peptide nucleic acid (PNA) sequence or a locked nucleic acid (LNA) sequence. In certain embodiments, the nucleic acid polymer carrier molecules are single- or multi-stranded, natural or synthetic DNA or RNA oligonucleotides, or DNA/RNA hybrids, or incorporating an unusual linker such as morpholine derivatized phosphates (AntiVirals, Inc., Corvallis Oreg.), or peptide nucleic acids such as N-(2-aminoethyl)glycine units, where the nucleic acid contains fewer than 50 nucleotides, more typically fewer than 25 nucleotides.

The carrier molecules used in the methods and composition described herein can also contain a carbohydrate or polyol, including a polysaccharide, such as dextran, FICOLL, heparin, glycogen, amylopectin, mannan, inulin, starch, agarose and cellulose, or a polymer such as a poly (ethylene glycol). In certain embodiments, the polysaccharide carrier molecule includes dextran, agarose or FICOLL.

The carrier molecules used in the methods and composition described herein can also include a lipid including, but not limited to, glycolipids, phospholipids, and sphingolipids. In certain embodiments, such lipids contain 6-25 carbons. In other embodiments, the carrier molecules include a lipid vesicle, such as a liposome,

The carrier molecules used in the methods and composition described herein can also be a cell, cellular systems, cellular fragment, or subcellular particles, including virus particles, bacterial particles, virus components, biological cells (such as animal cells, plant cells, bacteria, or yeast), or cellular components. Non-limiting examples of such cellular components that are useful as carrier molecules in the methods and composition described herein include lysosomes, endosomes, cytoplasm, nuclei, histones, mitochondria, Golgi apparatus, endoplasmic reticulum and vacuoles.

The carrier molecules used in the methods and composition described herein can also non-covalently associate with organic or inorganic materials.

The carrier molecules used in the methods and composition described herein can also include a specific binding pair member wherein the nucleic acid can be conjugated to a specific binding pair member and used in the formation of a bound pair. In certain embodiments, the presence of a labeled specific binding pair member indicates the location of the complementary member of that specific binding pair; each specific binding pair member having an area on the surface or in a cavity which specifically binds to, and is complementary with, a particular spatial and polar organization of the other. In certain embodiments, the labels described herein function as a reporter molecule for the specific binding pair. Exemplary binding pairs are set forth in Table 1.

TABLE 1
Representative Specific Binding Pairs

| | |
|---|---|
| antigen | antibody |
| biotin | avidin (or streptavidin or anti-biotin) |
| IgG* | protein A or protein G |
| drug | drug receptor |
| folate | folate binding protein |
| toxin | toxin receptor |
| carbohydrate | lectin or carbohydrate receptor |
| peptide | peptide receptor |
| protein | protein receptor |
| enzyme substrate | enzyme |
| DNA (RNA) | cDNA (cRNA)^{†} |
| hormone | hormone receptor |
| ion | chelator |

*IgG is an immunoglobulin
^{†}cDNA and cRNA are the complementary strands used for hybridization

In a particular aspect, the carrier molecule used in the methods and compositions described herein, is an antibody fragment, such as, but not limited to, anti-Fc, an anti-Fc isotype, anti-J chain, anti-kappa light chain, anti-lambda light chain, or a single-chain fragment variable protein; or a non-antibody peptide or protein, such as, for example but not limited to, soluble Fc receptor, protein G, protein A, protein L, lectins, or a fragment thereof. In one aspect the carrier molecule is a Fab fragment specific to the Fc portion of the target-binding antibody or to an isotype of the Fc portion of the target-binding antibody (U.S. Pat. No. 8,323,903). The monovalent Fab fragments are typically produced from either murine monoclonal antibodies or polyclonal antibodies generated in a variety of animals, for example but not limited to, rabbit or goat. These fragments can be generated from any isotype such as murine IgM, IgG₁, IgG₂ₐ, IgG_{2b} or IgG₃.

In alternative embodiments, a non-antibody protein or peptide such as protein G, or other suitable proteins, can be used alone or coupled with albumin. Preferred albumins include human and bovine serum albumins or ovalbumin. Proteins A, G and L are defined to include those proteins known to one skilled in the art or derivatives thereof that comprise at least one binding domain for IgG, i.e. proteins that have affinity for IgG. These proteins can be modified but do not need to be and are conjugated to a reactive moiety in the same manner as the other carrier molecules described.

In another aspect, the carrier molecules used in the methods and compositions described herein, can be whole intact antibodies. Antibody is a term of the art denoting the soluble substance or molecule secreted or produced by an animal in response to an antigen, and which has the particular property of combining specifically with the antigen that induced its formation. Antibodies themselves also serve are antigens or immunogens because they are glycoproteins and therefore are used to generate anti-species antibodies. Antibodies, also known as immunoglobulins, are classified into five distinct classes—IgG, IgA, IgM, IgD, and IgE. The basic IgG immunoglobulin structure consists of two identical light polypeptide chains and two identical heavy polypeptide chains (linked together by disulfide bonds).

When IgG is treated with the enzyme papain a monovalent antigen-binding fragment can be isolated, referred herein to as a Fab fragment. When IgG is treated with pepsin (another proteolytic enzyme), a larger fragment is produced, F(ab′)₂. This fragment can be split in half by treating with a mild reducing buffer that results in the monovalent Fab′ fragment. The Fab′ fragment is slightly larger than the Fab and contains one or more free sulfhydryls from the hinge region (which are not found in the smaller Fab fragment). The term “antibody fragment” is used herein to define the Fab′, F(ab′)₂ and Fab portions of the antibody. It is well known in the art to treat antibody molecules with pepsin and papain in order to produce antibody fragments (Gorevic et al., Meth. Enzymol., 116:3 (1985)).

The monovalent Fab fragments used as carrier molecules in the methods and compositions described herein are produced from either murine monoclonal antibodies or polyclonal antibodies generated in a variety of animals that have been immunized with a foreign antibody or fragment thereof (U.S. Pat. No. 4,196,265 discloses a method of producing monoclonal antibodies). Typically, secondary antibodies are derived from a polyclonal antibody that has been produced in a rabbit or goat but any animal known to one skilled in the art to produce polyclonal antibodies can be used to generate anti-species antibodies. The term “primary antibody” describes an antibody that binds directly to the antigen as opposed to a “secondary antibody” that binds to a region of the primary antibody. Monoclonal antibodies are equal, and in some cases, preferred over polyclonal antibodies provided that the ligand-binding antibody is compatible with the monoclonal antibodies that are typically produced from murine hybridoma cell lines using methods well known to one skilled in the art.

In one aspect the antibodies used as carrier molecules in the methods and compositions described herein are generated against only the Fc region of a foreign antibody. Essentially, the animal is immunized with only the Fc region fragment of a foreign antibody, such as murine. The polyclonal antibodies are collected from subsequent bleeds, digested with an enzyme, pepsin or papain, to produce monovalent fragments. The fragments are then affinity purified on a column comprising whole immunoglobulin protein that the animal was immunized against or just the Fc fragments.

Solid Supports:

In an aspect of the methods and composition described herein, the modified nucleic acids can be covalently conjugated to a solid support. In certain embodiments, the modified nucleic acids contain at least one alkyne moiety capable of reacting with a solid support containing an azide moiety.

A variety of solid supports can be used in the methods and compositions described herein. Such solid supports are not limited to a specific type of support, and therefore a large number of supports are available and are known to one of ordinary skill in the art. Such solid supports include, but are not limited to, solid and semi-solid matrixes, such as aerogels and hydrogels, resins, beads, biochips (including thin film coated biochips), microfluidic chip, a silicon chip, multi-well plates (also referred to as microtitre plates or microplates), membranes, conducting and nonconducting metals, glass (including microscope slides) and magnetic supports. Other non-limiting examples of solid supports used in the methods and compositions described herein include silica gels, polymeric membranes, particles, derivatized plastic films, derivatized glass, derivatized silica, glass beads, cotton, plastic beads, alumina gels, polysaccharides such as Sepharose, poly (acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like. In certain embodiments, the solid supports used in the methods and compositions described herein are substantially insoluble in liquid phases.

In certain embodiments, the solid support may include a solid support reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, wherein such functional groups are used to covalently attach the azide containing nucleic acids described herein. In other embodiments, the solid support may include a solid support reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, wherein such functional groups are used to covalently attach the alkyne-containing nucleic acids described herein. In still other embodiments, the solid support may include a solid support reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, wherein such functional groups are used to covalently attach the phosphine-containing nucleic acids described herein. In other embodiments, the solid supports include azide, alkyne or phosphine functional groups to covalently attach nucleic acids modified with azide, alkyne or phosphine moieties.

A suitable solid phase support used in the methods and compositions described herein, can be selected on the basis of desired end use and suitability for various synthetic protocols. By way of example only, where amide bond formation is desirable to attach the modified nucleic acids described herein to the solid support, resins generally useful in peptide synthesis may be employed, such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TentaGel™, Rapp Polymere, Tubingen, Germany), polydimethyl-acrylamide resin (available from Milligen/Biosearch, California), or PEGA beads (obtained from Polymer Laboratories). In certain embodiments, the modified nucleic acids described herein are deposited onto a solid support in an array format. In certain embodiments, such deposition is accomplished by direct surface contact between the support surface and a delivery mechanism, such as a pin or a capillary, or by inkjet technologies which utilize piezoelectric and other forms of propulsion to transfer liquids from miniature nozzles to solid surfaces. In the case of contact printing, robotic control systems and multiplexed printheads allow automated microarray fabrication. For contactless deposition by piezoelectric propulsion technologies, robotic systems also allow for automatic microarray fabrication using either continuous or drop-on-demand devices.

Kits:

According to certain embodiments of the present disclosure, kits are provided wherein the kits comprise:
an alkynyl-modified nucleoside analogue;
an adapter linker of structural formula (I);
a detectable label comprising a cycloalkene group; and
instructions for use according to the methods disclosed herein.

In certain embodiments, the kits further comprise a terminal deoxynucleotidyl transferase (TdT). In certain embodiments, the kits further comprise copper in the Cu(I) reduction state. In certain embodiments, the Cu(I) is a cuprous salt. In certain embodiments, the cuprous salt is a cuprous halide. In certain embodiments, the kits further comprise copper in the Cu(II) reduction state and a reducing agent. In certain embodiments, the Cu(II) is Cu(NO₃)₂, Cu(OAc)₂ or CuSO₄. In certain embodiments, the kits further comprise a copper chelator.

In certain embodiments, the detectable label is a colorimetric label. In certain embodiments, the kits are for labeling nucleic acid polymers. In certain embodiments, the kits are for measuring cellular nucleic acid synthesis. In certain embodiments, the kits are for measuring cellular proliferation. In certain embodiments, the kits are for identifying a test agent that perturbs cellular proliferation. In certain embodiments, the kits are for detecting apoptosis.

According to certain embodiments of the present disclosure, kits are provided, wherein the kits comprise:
an alkynyl-modified nucleoside analogue;
an azide-modified fluorescent dye;
an anti-fluorescent dye antibody that binds to the azide-modified fluorescent dye;
a secondary antibody conjugated to a detectable label, wherein the secondary antibody binds to the anti-fluorescent dye antibody; and
instructions for use according to the methods provided herein.

In certain embodiments, the kits further comprise a terminal deoxynucleotidyl transferase (TdT). In certain embodiments, the kits further comprise copper in the Cu(I) reduction state. In certain embodiments, the Cu(I) is a cuprous salt. In certain embodiments, the cuprous salt is a cuprous halide. In certain embodiments, the kits further comprise copper in the Cu(II) reduction state and a reducing agent. In certain embodiment, the Cu(II) is Cu(NO₃)₂, Cu(OAc)₂ or CuSO₄. In certain embodiments, the kits further comprise a copper chelator.

In certain embodiments, the detectable label is a colorimetric label. In certain embodiments, the kits are for labeling nucleic acid polymers. In certain embodiments, the kits are for measuring cellular nucleic acid synthesis. In certain embodiments, the kits are for measuring cellular proliferation. In certain embodiments, the kits are for identifying a test agent that perturbs cellular proliferation. In certain embodiments, the kits are for detecting apoptosis.

According to certain embodiments of the present disclosure, kits are provided, wherein the kits comprise:
an alkynyl-modified nucleoside analogue;
an azide-modified fluorescent dye;
an anti-fluorescent dye antibody conjugated to a detectable label, wherein the antibody binds to the azide-modified fluorescent dye; and
instructions for use according to the methods provided herein.

In certain embodiments, the kits further comprise a terminal deoxynucleotidyl transferase (TdT). In certain embodiments, the kits further comprise copper in the Cu(I) reduction state. In certain embodiments, the Cu(I) is a cuprous salt. In certain embodiments, the cuprous salt is a cuprous halide. In certain embodiments, the kits further comprise copper in the Cu(II) reduction state and a reducing agent. In certain embodiments, the Cu(II) is Cu(NO₃)₂, Cu(OAc)₂ or CuSO₄. In certain embodiments, the kits further comprise a copper chelator.

In certain embodiments, the detectable label is a colorimetric label. In certain embodiments, the kits are for labeling nucleic acid polymers. In certain embodiments, the kits are for measuring cellular nucleic acid synthesis. In certain embodiments, the kits are for measuring cellular proliferation. In certain embodiments, the kits are for identifying a test agent that perturbs cellular proliferation. In certain embodiments, the kits are for detecting apoptosis.

According to certain embodiments of the present disclosure kits are provided, wherein the kits comprise:
an alkynyl-modified nucleoside analogue;
an azide-modified biotin;
a (strept)avidin conjugated to a detectable label; and
instructions for use according to the methods provided herein.

In certain embodiments, the kits further comprise a terminal deoxynucleotidyl transferase (TdT). In certain embodiments, the kits further comprise copper in the Cu(I) reduction state. In certain embodiments, the Cu(I) is a cuprous salt. In certain embodiments, the cuprous salt is a cuprous halide. In certain embodiments, the kits further comprise copper in the Cu(II) reduction state and a reducing agent. In certain embodiments, the Cu(II) is Cu(NO₃)₂, Cu(OAc)₂ or CuSO₄. In certain embodiments, the kits further comprise a copper chelator.

In certain embodiments, the detectable label is a colorimetric label. In certain embodiments, the kits are for labeling nucleic acid polymers. In certain embodiments, the kits are for measuring cellular nucleic acid synthesis. In certain embodiments, the kits are for measuring cellular proliferation. In certain embodiments, the kits are for identifying a test agent that perturbs cellular proliferation. In certain embodiments, the kits are for detecting apoptosis.

In certain embodiments, the kits further comprise one or more of the following: a buffering agent, a purification medium, a vial comprising the sample, or an organic solvent.

The kits may further comprise one or more pieces of equipment to administer the components of the kits including, but not limited to, syringes, pipettes, pipette bulbs, spatulas, vials, syringe needles, and various combinations thereof.

In certain embodiments, the kits provided herein comprise indicator solutions or indicator “dipsticks”, blotters, culture media, cuvettes, and the like. In certain embodiments, the kits provided herein comprise indicator cartridges (where a kit component is bound to a solid support) for use in an automated detector. In certain embodiments, the kits provided herein further comprise molecular weight markers, wherein said markers are selected from phosphorylated and non-phosphorylated polypeptides, calcium-binding and non-calcium binding polypeptides, sulfonated and non-sulfonated polypeptides, and sialylated and non-sialylated polypeptides. In certain embodiments, the kits provided herein further comprise a member selected from a fixing solution, a detection reagent, a standard, a wash solution, and combinations thereof.

A detailed description of the present teachings having been provided above, the following examples are given for the purpose of illustrating the present teachings and shall not be construed as being a limitation on the scope of the present teachings or claims.

### EXAMPLES

The following examples describe some of the preferred embodiments of the present disclosure. However, it should be understood that these examples are for illustrative purposes only and are not meant to limit the scope of the present disclosure.

### Example 1

### Double Click Reaction

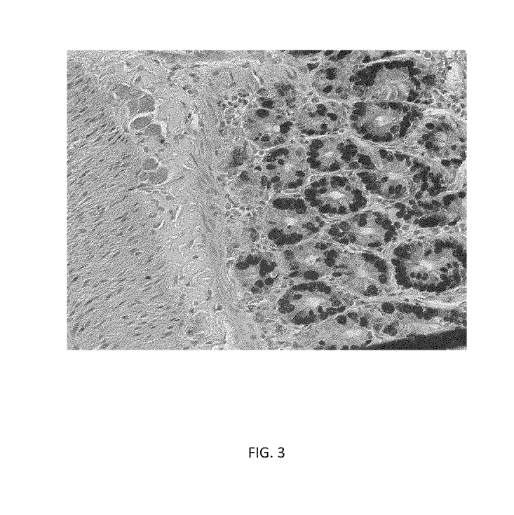

Formaldehyde fixed paraffin embedded tissue (FFPE), that was previously labeled with EdU, was deparaffinized and rehydrated using standard xylene and graded ethanol washes followed by peroxidase quenching for 10 minutes using 3% hydrogen peroxidase (H₂O₂) in phosphate buffered saline (PBS). DNA denaturation using 2 N HCl followed by neutralization with sodium borate then proteinase K digestion was used to prepared the tissue for double click labeling. Incubation for 30 minutes in copper-mediated click reaction attached a tetrazine-azide bi-functional linker according to Structural Formula (I) to the EdU incorporated within the DNA. A PBS rinse followed by one hour incubation with trans-cyclooctene-labeled HRP in neutral pH resulted in the covalent attachment of HRP to the EdU tetrazine complex. The tissue was rinsed in PBS prior to treatment with DAB to develop the dark brown color in proliferating cells. The tissue was counterstained with Mayer's hematoxylin and then blued with tap water (see, FIG. 3).

### Example 2

### Copper-Based Click Reaction of a Fluorescent Azide Intermediate

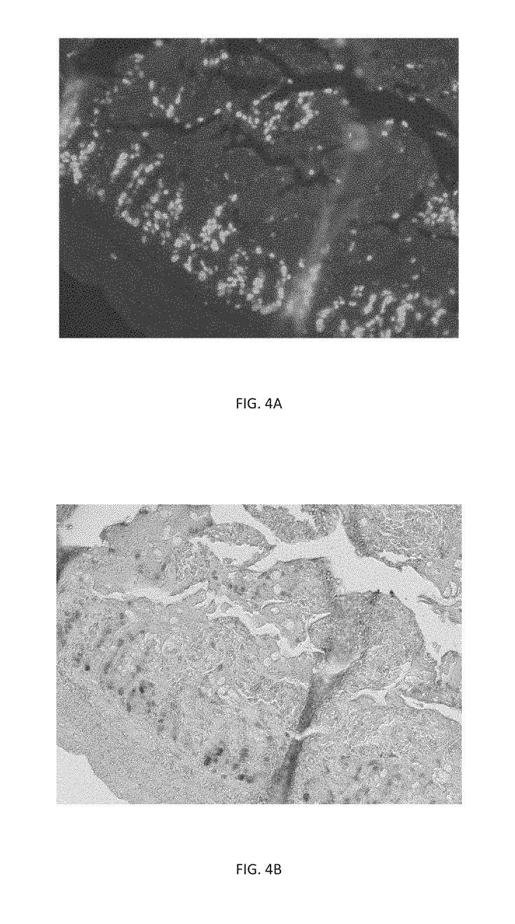

Formaldehyde fixed paraffin embedded tissue (FFPE), that was previously labeled with EdU, was deparaffinized and rehydrated using standard xylene and graded ethanol washes followed by peroxidase quenching for 10 minutes using 3% hydrogen peroxidase (H₂O₂) in phosphate buffered saline (PBS). DNA denaturation using 2 N HCl followed by neutralization with sodium borate then proteinase K digestion and a short fix with 3.7% paraformaldehyde was used to prepared the tissue for OREGON GREEN™-azide click labeling. Incubation for 30 minutes in copper mediated click reaction attached an OREGON GREEN™ dye to the incorporated EdU within the DNA. After rinsing, the tissue was imaged as a wet mount using a fluorescence microscope fitted with a FITC filter set demonstrating the intermediate fluorescence signal visible used as verification within the protocol (see, FIG. 4A). The tissue was blocked with 3% bovine serum albumin+5% normal goat serum in PBS rinse followed by one hour incubation with the rabbit anti-OREGON GREEN antibody. The tissue was rinsed in PBS and then incubated with goat-anti-rabbit HRP conjugate for 30 minutes at room temperature. After rinsing with PBS the tissue was incubated with DAB to develop the dark brown color in proliferating cells. The tissue was again imaged using the same magnification of the same field using bright field microscopy to demonstrate the conversion of the fluorescent signal to a colorimetric based signal (see, FIG. 4B).

### Example 3

### Copper-Based Click Reaction of a Biotin Azide

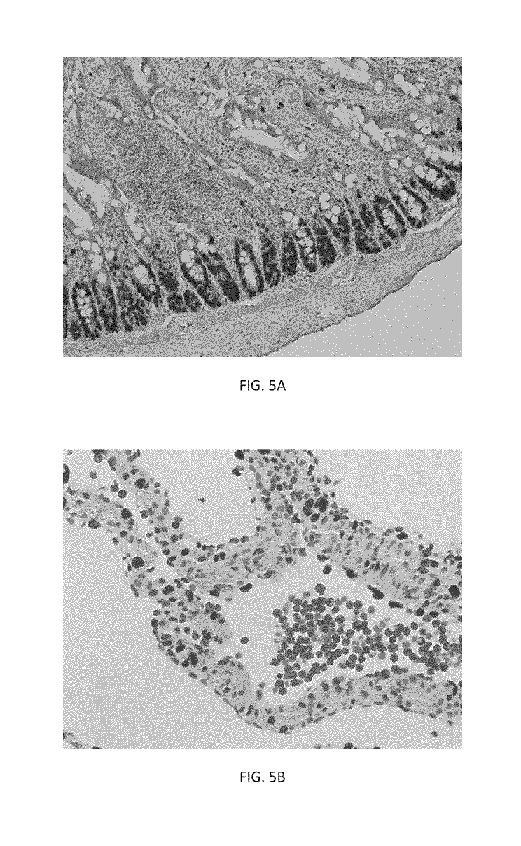

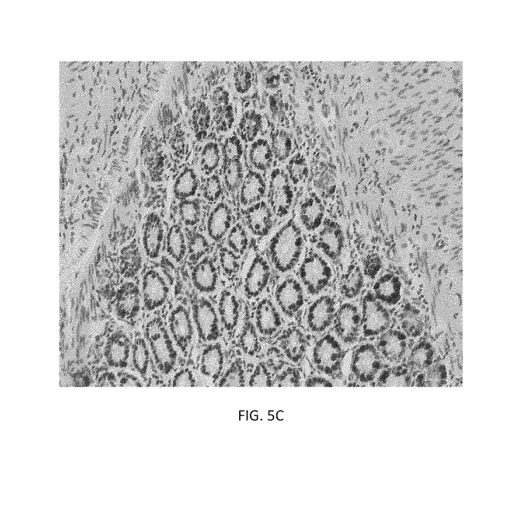

Formaldehyde fixed paraffin embedded tissue (FFPE), that was previously labeled with EdU, was deparaffinized and rehydrated using standard xylene and graded ethanol washes followed by peroxidase quenching for 10 minutes using 3% hydrogen peroxidase (H₂O₂) in phosphate buffered saline (PBS). Nuclear accessibility was obtained using trypsin digestion for 20 minutes followed by heat inactivated epitope retrieval (HIER) at 95° C. in Tris pH 8.0 for 20 minute followed by cooling to room temperature. Incubation for 30 minutes in copper mediated click reaction with a biotin azide attached a biotin to the incorporated EdU within the DNA. The tissue was blocked with 3% bovine serum albumin+5% normal goat serum in PBS followed by 30 minute incubation with the (strept)avidin-HRP conjugate. After rinsing with PBS the tissue was incubated with DAB to develop the dark brown color in proliferating cells. The tissue was counterstained with Mayer's hematoxylin and then blued with tap water (see, FIG. 5A).

Formaldehyde fixed paraffin embedded tissue (FFPE), that was previously labeled with EdU, was deparaffinized and rehydrated using standard xylene and graded ethanol washes followed by peroxidase quenching for 10 minutes using 3% hydrogen peroxidase (H₂O₂) in phosphate buffered saline (PBS). Nuclear accessibility was obtained using trypsin digestion for 20 minutes. No HIER antigen retrieval step was performed prior to incubation for 30 minutes in copper mediated click reaction with a biotin azide to attach a biotin to the incorporated EdU within the DNA. The tissue was blocked with 3% bovine serum albumin+5% normal goat serum in PBS followed by 30 minute incubation with the (strept)avidin-HRP conjugate. After rinsing with PBS the tissue was incubated with DAB to develop the dark brown color in proliferating cells. The tissue was counterstained with Russell-Movat pentachrome stain (see, FIG. 5B).

Formaldehyde fixed paraffin embedded tissue (FFPE), that was previously labeled with EdU, was deparaffinized and rehydrated using standard xylene and graded ethanol washes followed by peroxidase quenching for 10 minutes using 3% hydrogen peroxidase (H₂O₂) in phosphate buffered saline (PBS). Nuclear accessibility was obtained using trypsin digestion for 30 minutes prior to PBS rinse and incubation for 30 minutes in copper mediated click reaction to attach an OREGON GREEN™ dye to the incorporated EdU within the DNA. The tissue was blocked with 3% bovine serum albumin+5% normal goat serum in PBS and rinsed followed by one hour incubation with the rabbit anti-OREGON GREEN antibody. The tissue was rinsed in PBS and then incubated with goat-anti-rabbit HRP conjugate for 30 minutes at room temperature. After rinsing with PBS the tissue was incubated with DAB to develop the dark brown color in proliferating cells. The tissue was counterstained with hematoxylin and then blued with tap water. (see, FIG. 5C).

### Example 4

### Optimizing Workflow and Signal/Noise Ratio:

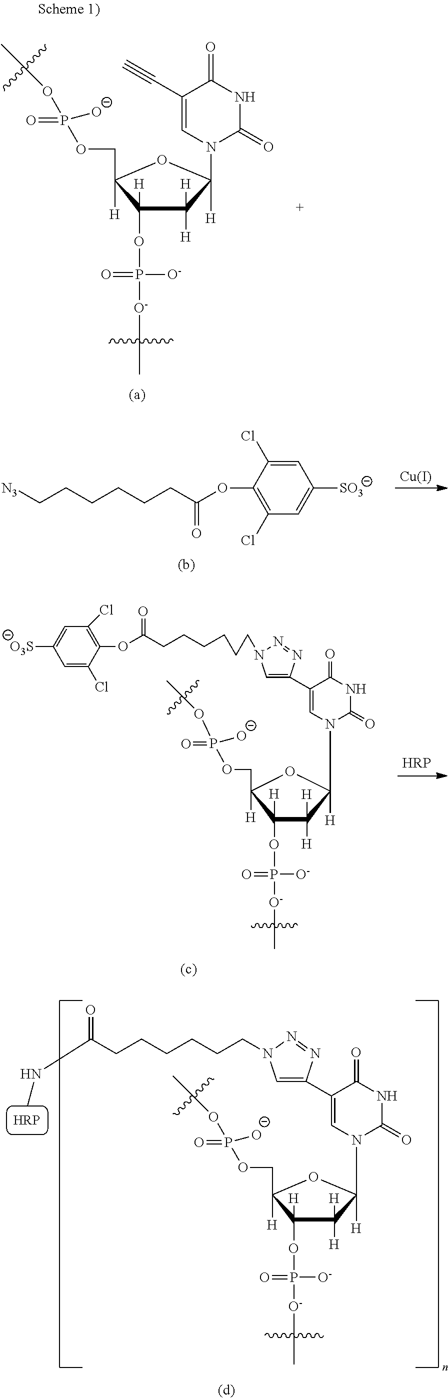

A sample containing nucleic acid (a) in which one or more 5-ethynyl-2-deoxyuridine moieties has been incorporated is treated with a 10× molar excess of the 2,6-dichloro-4-sulfophenyl (SDP) ester of 7-azidoheptanoic acid (b) in aqueous media at pH 7-8, in the presence of a 0.5× molar equivalent of copper(I) ion. After a 15 minute reaction period at ambient temperature, the sample is rinsed in PBS to remove excess (b) and copper(I) ion. The sample containing the SDP ester form of the labeled nucleic acid (c) is reacted with excess horseradish peroxidase (HRP, Thermo Fisher Scientific product 012001) in aqueous bicarbonate buffer at pH 8.5 for 30-60 minutes. The sample is rinsed with PBS, and DAB signal detected as described in Example 1.

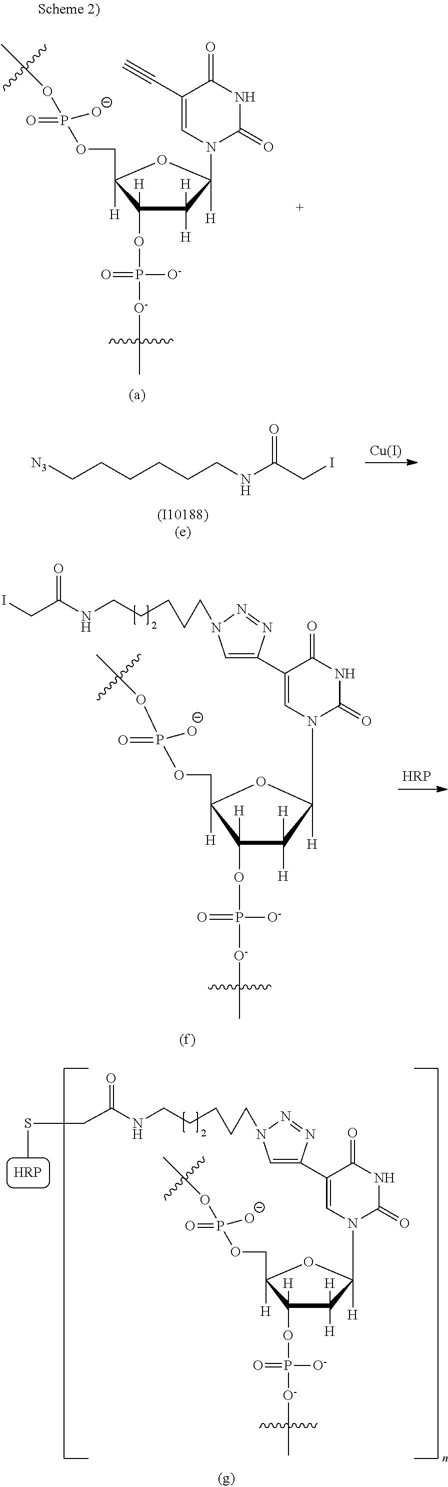

A sample containing nucleic acid (a) in which one or more 5-ethynyl-2-deoxyuridine moieties has been incorporated is treated with a 10× molar excess of the iodoacetamide of 6-azidohexylamine (e) in aqueous media at pH 7-8, in the presence of a 0.5× molar equivalent of copper(I) ion. After a 15 minute reaction period at ambient temperature, the sample is rinsed in PBS to remove excess (e) and copper(I) ion. The sample containing the iodoacetamide form of the labeled nucleic acid (f) is reacted with excess horseradish peroxidase (HRP, Thermo Fisher Scientific product 012001) in aqueous bicarbonate buffer at pH 8.5 for 120 minutes. The sample containing (g) is rinsed with PBS, and DAB signal detected as described in Example 1.

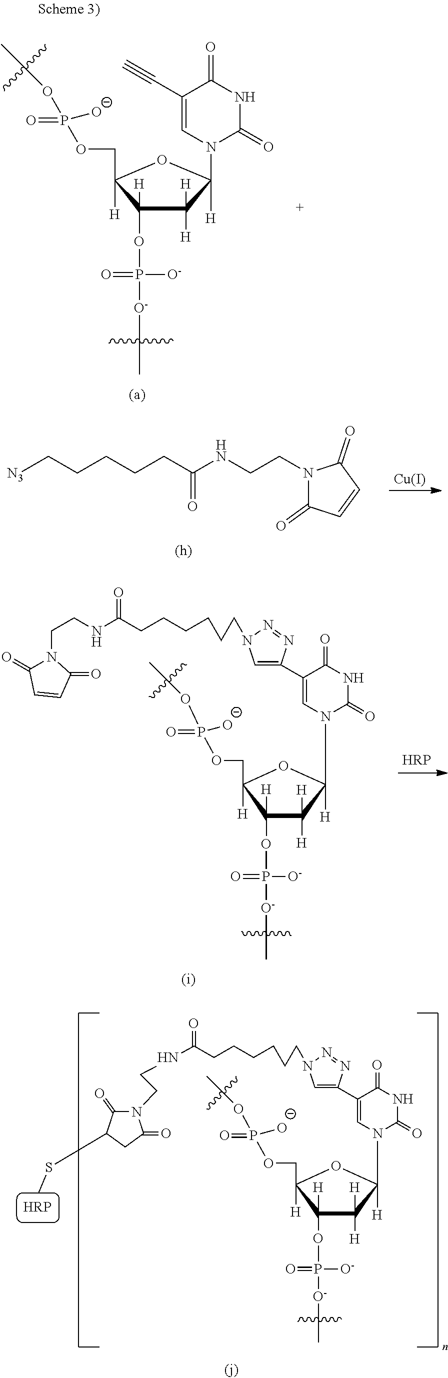

A sample containing nucleic acid (a) in which one or more 5-ethynyl-2-deoxyuridine moieties has been incorporated is treated with a 10× molar excess of the azido maleimide (h) in aqueous media at pH 7-8, in the presence of a 0.5× molar equivalent of copper(I) ion. After a 15 minute reaction period at ambient temperature, the sample is rinsed in PBS to remove excess (h) and copper(I) ion. The sample containing the maleimide form of the labeled nucleic acid (i) is reacted with excess horseradish peroxidase (HRP, Thermo Fisher Scientific product 012001) in aqueous bicarbonate buffer at pH 8.5 for 120 minutes. The sample containing (j) is rinsed with PBS, and DAB signal detected as described in Example 1.

### Example 5

### Comparison of Nucleic Acid Labeling with BrdU Versus EdU

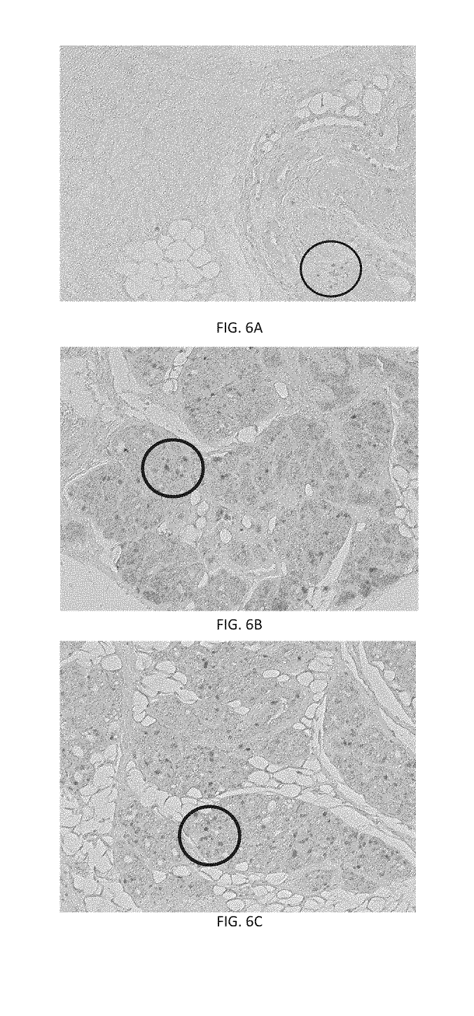

Signal intensity of BrdU was compared to EdU using mammary epithelial tissue from either BrdU or EdU-pulsed rats. Formaldehyde fixed paraffin embedded tissue (FFPE) was deparaffinized and rehydrated using standard xylene and graded ethanol washes followed by peroxidase quenching for 10 minutes using 3% hydrogen peroxidase (H₂O₂) in phosphate buffered saline (PBS). BrdU detection was performed following instructions of a commercially available BrdU kit which utilized trypsin digestion and HCl for DNA revelation followed by a protein blocking step and subsequent one hour incubation with a biotinylated anti-BrdU antibody. Incubation with streptavidin-HRP reagent followed by a wash step and incubation with DAB substrate and buffer resulted in deposition of the chromophore at the site of BrdU incorporation. EdU detection in EdU pulsed tissue was performed as indicated in FIG. 2A, by first treating with trypsin followed by a copper(I) catalyzed click reaction with biotin azide for 30 minutes followed by wash step and incubation with streptavidin-HRP. After another wash step and incubation with DAB substrate, chromophore deposition occurred at the sites of EdU incorporation. The circled areas indicate signal derived from DAB showing clustering of proliferation from BrdU (FIG. 6A) compared to EdU (FIG. 6B and FIG. 6C).

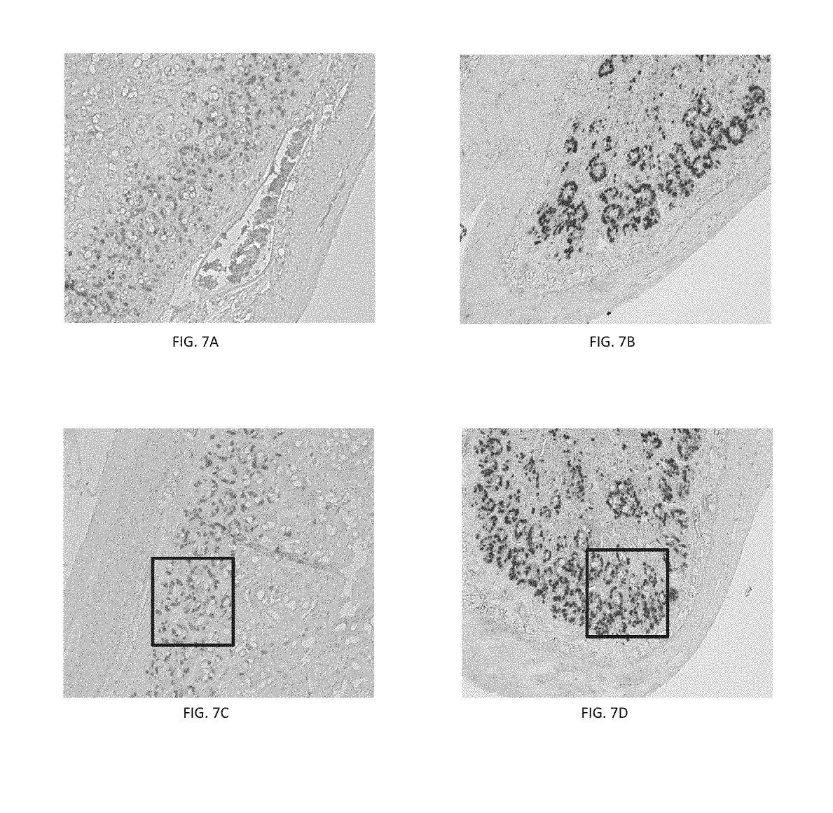

Signal intensity of BrdU was compared to EdU using intestinal tissue from either BrdU or EdU-pulsed rats. Formaldehyde fixed paraffin embedded tissue (FFPE) was deparaffinized and rehydrated using standard xylene and graded ethanol washes followed by peroxidase quenching for 10 minutes using 3% hydrogen peroxidase (H₂O₂) in phosphate buffered saline (PBS). BrdU detection was performed following instructions of a commercially available BrdU kit which utilized trypsin digestion and HCl for DNA revelation followed by a protein blocking step and subsequent one hour incubation with a biotinylated anti-BrdU antibody. Incubation with streptavidin-HRP reagent followed by a wash step and incubation with DAB substrate and buffer resulted in deposition of chromophore at the site of BrdU incorporation. EdU detection in EdU pulsed tissue was performed as outlined in FIG. 2A, by first treating with trypsin followed by a copper(I) catalyzed click reaction with biotin azide for 30 minutes followed by wash step and incubation with streptavidin-HRP. After another wash step and incubation with DAB substrate and buffer resulting in deposition of a chromophore at the site of EdU incorporation. Representative images showing BrdU labeling (FIG. 7A and FIG. 7C) compared to EdU labeling (FIG. 7B and FIG. 7D). The rectangular marked areas within FIG. 7C and FIG. 7D indicate representative areas of highly proliferative regions of intestinal tissue comparing BrdU labeled tissue (FIG. 7C) to EdU labeled tissue (FIG. 7D).

### Example 6

### TUNEL Assay

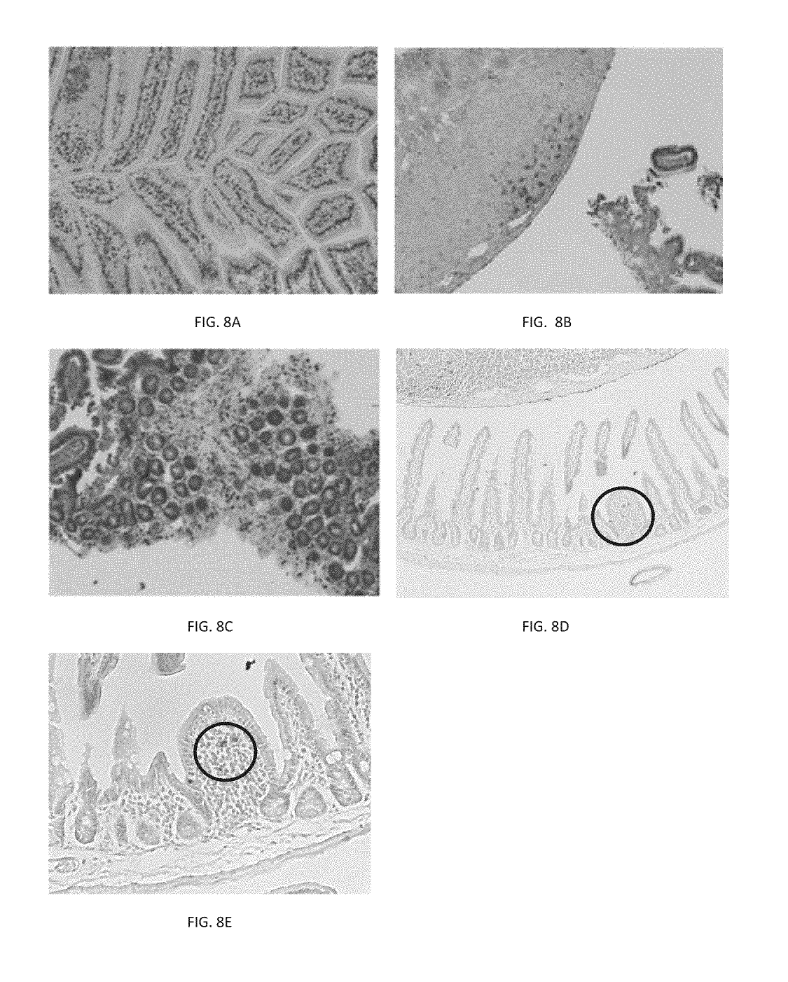

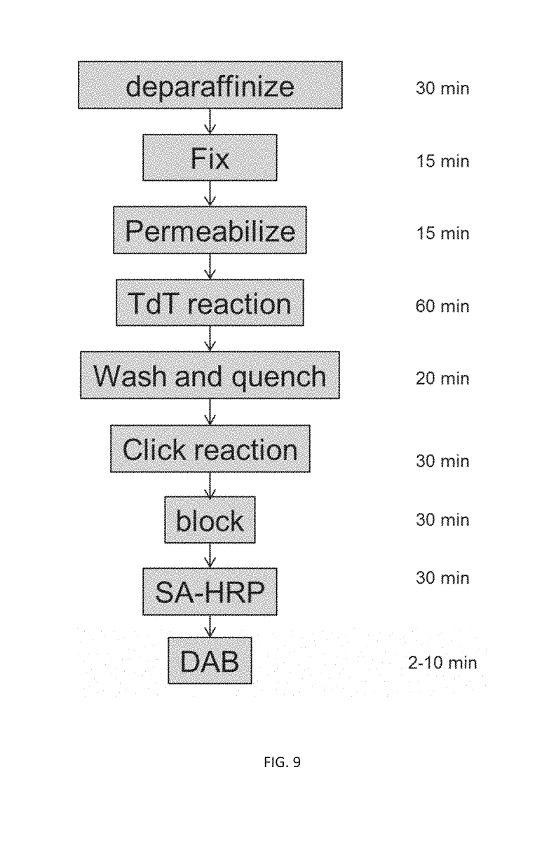

The TUNEL Assay used in this example is outlined in FIG. 9. Formaldehyde fixed paraffin embedded (FFPE) mouse intestine tissue was deparaffinized and rehydrated using standard xylene and graded ethanol washes. The tissues were then re-fixed for 15 minutes with 4% paraformaldehyde in phosphate buffered saline (PBS), followed by Proteinase K digestion for 15 minutes to gain nuclear access, and followed by an additional 4% paraformaldehyde in PBS fixation. Incubation in TdT buffer for 10 minutes was followed by a 60 minute incubation with TdT and an EdUTP nucleotide mixture in TdT buffer to incorporate the terminal alkyne to the 3′OH ends of DNA. The TdT reaction was quenched in 10×SSC buffer for 15 minutes, followed by peroxidase quenching for 5 minutes using 3% hydrogen peroxidase (H₂O₂) in PBS. Incubation for 30 minutes in copper mediated click reaction attached a biotin to the incorporated alkyne within the DNA. The tissue was blocked with 5 mg/mL bovine serum albumin+0.1% Triton in PBS followed by 30 minute incubation with the streptavidin-HRP conjugate. After rinsing with PBS the tissue was incubated with DAB to develop the dark brown color in apoptotic cells. The tissue was counterstained with Mayer's hematoxylin and then blued with tap water. FIG. 8A shows mouse intestine treated with DNase, FIG. 8B shows mouse intestine without DNase treatment. FIG. 8C shows mouse intestine stained only with H&E. FIGS. 8D and 8E show two magnifications of FFPE mouse intestine tissue that was treated in the same manner as above, with the exception that the tissue was counterstained with Methyl Green. FIG. 8D shows a 20× magnification and FIG. 8E shows a 40× magnification with the apoptotic staining circled.

1-58. (canceled)

59. A method of measuring cellular proliferation in an organism, the method comprising:
a) administering to an organism an effective amount of an alkynyl-modified nucleoside analogue, such that the alkynyl-modified nucleoside analogue is incorporated into DNA of cells of the organism;
b) contacting at least one cell of the organism with an azide-modified biotin under conditions such that the alkynyl moiety of the alkynyl-modified nucleoside analogue forms a covalent link with the azide moiety of the biotin;
c) contacting the at least one cell of the organism with a (strept)avidin conjugated to a detectable label; and
d) measuring the amount of detectable label, wherein the amount of label indicates the extent of cellular proliferation.

60. (canceled)

61. A method for identifying an agent that perturbs cellular proliferation in an organism, the method comprising:
a) exposing an organism to a test agent;
b) administering to the organism an effective amount of an alkynyl-modified nucleoside analogue, such that the alkynyl-modified nucleoside analogue is incorporated into DNA of cells of the organism;
c) contacting at least one cell of the organism with an azide-modified biotin under conditions such that the alkynyl moiety of the alkynyl-modified nucleoside analogue forms a covalent link with the azide moiety of the biotin;
d) contacting the at least one cell of the organism with a (strept)avidin conjugated to a detectable label;
e) measuring the amount of detectable label, wherein the amount of label indicates the extent of cellular proliferation; and
f) identifying the test agent as an agent that perturbs cellular proliferation if the amount of label measured in step (e) is less than or greater than the amount of label measured in a control application in which the organism is not exposed to the test agent.

62-63. (canceled)

64. A method of detecting apoptosis, the method comprising:
a) contacting a cell with an effective amount of an alkynyl-modified nucleotide analogue and a terminal deoxynucleotidyl transferase (TdT), such that the alkynyl-modified nucleotide analogue is incorporated into DNA of the cell;
b) contacting the cell with an azide-modified biotin under conditions such that the alkynyl moiety of the alkynyl-modified nucleotide analogue forms a covalent link with the azide moiety of the biotin;
c) contacting the cell with a (strept)avidin conjugated to a detectable label; and
d) measuring the amount of detectable label, wherein the amount of label indicates the amount of apoptosis.

65. The method of claim 64, wherein the alkynyl-modified nucleotide analogue is an EdUTP, an EdATP, an EdCTP, an EdGTP or an EdTTP.

66. The method of claim 59, wherein the alkynyl-modified nucleoside analogue is an EdU or an EdC.

67. (canceled)

68. The method of claim 59, wherein the detectable label is a colorimetric label.

69. The method of claim 68, wherein the colorimetric label is selected from horseradish peroxidase, alkaline phosphatase, beta-galactosidase, glucose oxidase and beta-lactamase.

70. The method of claim 59, wherein the step of contacting the at least one cell of the organism with the azide-modified biotin is performed in the presence of copper in the Cu(I) reduction state.

71. The method of claim 59, wherein the step of contacting the at least one cell of the organism with the azide-modified biotin is performed in the presence of copper in the Cu(II) reduction state and a reducing agent.

72. The method of claim 59, wherein the step of contacting the at least one cell of the organism with the azide-modified biotin is performed in the presence of a copper chelator.

73. The method of claim 61, wherein the step of contacting the at least one cell of the organism with the azide-modified biotin is performed in the presence of copper in the Cu(I) reduction state.

74. The method of claim 61, wherein the step of contacting the at least one cell of the organism with the azide-modified biotin is performed in the presence of copper in the Cu(II) reduction state and a reducing agent.

75. The method of claim 61, wherein the step of contacting the at least one cell of the organism with the azide-modified biotin is performed in the presence of a copper chelator.

76. A kit comprising:
an alkynyl-modified nucleotide analogue;
an azide-modified biotin;
a (strept)avidin conjugated to a detectable label; and
instructions for use according to the method of claim 64.

77. The kit of claim 76, further comprising:
i) a terminal deoxynucleotidyl transferase; and/or
ii) copper in the Cu(I) reduction state; and/or
iii) copper in the Cu(II) reduction state and a reducing agent; and/or
iv) a copper chelator.

78. The method of claim 64, wherein the detectable label is a colorimetric label.

79. The method of claim 64, wherein the colorimetric label is selected from horseradish peroxidase, alkaline phosphatase, beta-galactosidase, glucose oxidase and beta-lactamase.

80. The method of claim 64, wherein the step of contacting the alkynyl-modified nucleic acid polymer with the azide-modified biotin is performed in the presence of copper in the Cu(II) reduction state and a reducing agent.

81. The method of claim 64, wherein the step of contacting the alkynyl-modified nucleic acid polymer with the azide-modified biotin is performed in the presence of a copper chelator.

82. The method of claim 61, wherein the detectable label is a colorimetric label.
